SEQUENCE LISTING (53 sequences)

SEQ ID NO: 1 - protein, 5 aa, Artificial Sequence
  Synthetic
SYWMH

SEQ ID NO: 2 - protein, 5 aa, Artificial Sequence
  Synthetic
SYWIH

SEQ ID NO: 3 - protein, 17 aa, Artificial Sequence
  Synthetic
EIIPIFGHTNYNEKFKS

SEQ ID NO: 4 - protein, 17 aa, Artificial Sequence
  Synthetic
EIIPIFGHTNYNERFQG

SEQ ID NO: 5 - protein, 14 aa, Artificial Sequence
  Synthetic
GGYYYYGSRDYFDY

SEQ ID NO: 6 - protein, 14 aa, Artificial Sequence
  Synthetic
GGYYYYFNSGTLDY

SEQ ID NO: 7 - protein, 14 aa, Artificial Sequence
  Synthetic
GGYYYYIGKGVLDY

SEQ ID NO: 8 - protein, 14 aa, Artificial Sequence
  Synthetic
GGYYYYPRFGWLDY

SEQ ID NO: 9 - protein, 14 aa, Artificial Sequence
  Synthetic
GGYYYYTGKGVLDY

SEQ ID NO: 10 - protein, 14 aa, Artificial Sequence
  Synthetic
GGYYYYDSNAVLDY

SEQ ID NO: 11 - protein, 14 aa, Artificial Sequence
  Synthetic
GGYYYYFHSGTLDY

SEQ ID NO: 12 - protein, 14 aa, Artificial Sequence
  Synthetic
GGYYYYFNKAVLDY

SEQ ID NO: 13 - protein, 14 aa, Artificial Sequence
  Synthetic
GGYYYYGGSGVLDY

SEQ ID NO: 14 - protein, 14 aa, Artificial Sequence
  Synthetic
GGYYYYPRQGFLDY

SEQ ID NO: 15 - protein, 14 aa, Artificial Sequence
  Synthetic
GGYYYYYNSGTLDY

SEQ ID NO: 16 - protein, 15 aa, Artificial Sequence
  Synthetic
KASQSVDYSGDSYMN

SEQ ID NO: 17 - protein, 15 aa, Artificial Sequence
  Synthetic
RASQSVDYSGDSYMN

SEQ ID NO: 18 - protein, 15 aa, Artificial Sequence
  Synthetic
RASQSVDYSGDSYLN

SEQ ID NO: 19 - protein, 7 aa, Artificial Sequence
  Synthetic
AASDLES

SEQ ID NO: 20 - protein, 9 aa, Artificial Sequence
  Synthetic
QQSHEDPFT

SEQ ID NO: 21 - protein, 9 aa, Artificial Sequence
  Synthetic
QQSYSTPFT

SEQ ID NO: 22 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSS

SEQ ID NO: 23 - protein, 123 aa, Artificial Sequence
  Synthetic
EVQLVESGGGLVKPGGSLRLSCAASGFTFSSYWMHWVRQAPGKGLEWVGEIIPIFGHTNYNERFQGRFTISLDNSKNTLYLQMGSLRAEDTAVYYCARGGYYYYGSRDYFDYWGQGTTVTVSS

SEQ ID NO: 24 - protein, 123 aa, Artificial Sequence
  Synthetic
EVQLVESGGGLVKPGGSLRLSCAASGFTFSSYWIHWVRQAPGKGLEWVGEIIPIFGHTNYNERFQGRFTISLDNSKNTLYLQMGSLRAEDTAVYYCARGGYYYYGSRDYFDYWGQGTTVTVSS

SEQ ID NO: 25 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFNSGTLDYWGQGTTVTVSS

SEQ ID NO: 26 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYIGKGVLDYWGQGTTVTVSS

SEQ ID NO: 27 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRFGWLDYWGQGTTVTVSS

SEQ ID NO: 28 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYTGKGVLDYWGQGTTVTVSS

SEQ ID NO: 29 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYDSNAVLDYWGQGTTVTVSS

SEQ ID NO: 30 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFHSGTLDYWGQGTTVTVSS

SEQ ID NO: 31 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYFNKAVLDYWGQGTTVTVSS

SEQ ID NO: 32 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGGSGVLDYWGQGTTVTVSS

SEQ ID NO: 33 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 34 - protein, 123 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYYNSGTLDYWGQGTTVTVSS

SEQ ID NO: 35 - protein, 111 aa, Artificial Sequence
  Synthetic
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 36 - protein, 111 aa, Artificial Sequence
  Synthetic
DIVMTQSPDSLAVSLGERATISCRASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 37 - protein, 111 aa, Artificial Sequence
  Synthetic
DIVMTQSPDSLAVSLGERATISCRASQSVDYSGDSYLNWYQQKPGQPPKLLIYAASDLESGIPARFSGSGSGTDFTLTISSLEPEDFATYYCQQSYSTPFTFGQGTKLEIK

SEQ ID NO: 38 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYGSRDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 39 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRFGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 40 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 41 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 42 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 43 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 44 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 45 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 46 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 47 - protein, 453 aa, Artificial Sequence
  Synthetic
  misc_feature (453)..(453): Xaa is a lysine (K) or is absent
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPGX

SEQ ID NO: 48 - protein, 452 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 452 aa, Artificial Sequence
  Synthetic
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 50 - protein, 218 aa, Artificial Sequence
  Synthetic
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 4 aa, Artificial Sequence
  Synthetic
ALAL

SEQ ID NO: 52 - protein, 4 aa, Artificial Sequence
  Synthetic
  MOD_RES (1)..(1): b-Ala
ALAL

SEQ ID NO: 53 - protein, 4 aa, Artificial Sequence
  Synthetic
GFLG

(19) United States
(12) Patent Application Publication
(10) Pub. No.: US 2024/0165256 A1
(43) Pub. Date: May 23, 2024

(54) METHODS FOR INCREASING EFFICACY OF IMMUNOCONJUGATES TARGETING ADAM9 FOR THE TREATMENT OF CANCER

(71) Applicants: IMMUNOGEN, INC., Waltham, MA (US); MACROGENICS, INC., Rockville, MD (US)

(72) Inventors: Eric Henry Westin, Waltham, MA (US); Callum Sloss, Waltham, MA (US); Krystal Marie Watkins, Waltham, MA (US); Deryk T. Loo, Rockville, CA (US); Juniper A. Scribner, Rockville, CA (US); Francine Zhifen Chen, Rockville, CA (US)

(21) Appl. No.: 18/280,896

(22) PCT Filed: Mar. 7, 2022
(86) PCT No.: PCT/US2022/019133

§ 371 Date: Sep. 7, 2023

Related U.S. Application Data
(60) Provisional application No. 63/157,954, filed on Mar. 8, 2021.

(51) Int. Cl. A61K 47/68; A61P 35/00; G01N 33/574

(52) U.S. Cl. CPC A61K 47/6851; A61K 47/68033; A61K 47/6889; A61P 35/00; G01N 33/57484; G01N 2333/96486

(57) ABSTRACT

The present disclosure provides methods of treating cancer in a subject who has an increased level of ADAM9 expression. The method comprises administering to the subject a therapeutically effective amount of an anti-ADAM9 immunoconjugate. Also provided is a method of increasing the efficacy of cancer treatment with an anti-ADAM9 immunoconjugate in a subject who has an increased level of ADAM9 expression.

### RELATED APPLICATION

This application claims the benefit of priority to U.S. Provisional Application No. 63/157,954, filed Mar. 8, 2021, the entire contents of which are incorporated herein by reference.

### FIELD OF THE INVENTION

The present invention generally relates to methods of increasing efficacy of the treatment of cancers with ADAM9 immunoconjugates comprising an antibody or fragment thereof capable of specifically binding to “Disintegrin and Metalloproteinase Domain-containing Protein 9” (“ADAM9”) conjugated to at least one pharmacological agent. More specifically, the invention relates to more effective treatment of patients susceptible to or diagnosed with cancer, in which the tumor cells overexpress ADAM9 as determined by an IHC assay, with an anti-ADAM9 immunonoconjugate.

### BACKGROUND OF THE INVENTION

ADAM9 is a member of the ADAM family of molecules. The expression of ADAM9 has been found to be relevant to disease, especially cancer. ADAM9 has been found to cleave and release a number of molecules with important roles in tumorigenesis and angiogenesis, such as TEK, KDR, EPHB4, CD40, VCAM1 and CDHS. ADAM9 is expressed by many types of tumor cells, including tumor cells of breast cancers, colon cancers, gastric cancers, gliomas, liver cancers, non-small cell lung cancers, melanomas, myelomas, pancreatic cancers and prostate cancers (Yoshimasu, T. et al. (2004) “Overexpression Of ADAM9 In Non-Small Cell Lung Cancer Correlates With Brain Metastasis,” Cancer Res. 64: 4190-4196; Peduto, L. et al. (2005) “Critical Function For ADAM9 In Mouse Prostate Cancer,” Cancer Res. 65: 9312-9319; Zigrino, P. et al. (2005) “ADAM-9 Expression And Regulation In Human Skin Melanoma And Melanoma Cell Lines,” Int. J. Cancer 116: 853-859; Fritzsche, F. R. et al. (2008) “ADAM9 Is Highly Expressed In Renal Cell Cancer And Is Associated With Tumour Progression,” BMC Cancer 8: 179: 1-9; Fry, J. L. et al. (2010) “Secreted And Membrane-Bound Isoforms Of Protease ADAM9 Have Opposing Effects On Breast Cancer Cell Migration,” Cancer Res. 70, 8187-8198; Chang, L. et al. (2016) “Combined Rnai Targeting Human Stat3 And ADAM9 As Gene Therapy For Non-Small Cell Lung Cancer,” Oncology Letters 11: 1242-1250; Fan, X. et al. (2016) “ADAM9 Expression Is Associate with Glioma Tumor Grade and Histological Type, and Acts as a Prognostic Factor in Lower-Grade Gliomas,” Int. J. Mol. Sci. 17: 1276:1-11).

Significantly, increased ADAM9 expression has been found to correlate positively with tumor malignancy and metastatic potential (Amendola, R. S. et al. (2015) “ADAM9 Disintegrin Domain Activates Human Neutrophils Through An Autocrine Circuit Involving Integrins And CXCR2,” J. Leukocyte Biol. 97(5): 951-962; Fan, X. et al. (2016) “ADAM9 Expression Is Associate with Glioma Tumor Grade and Histological Type, and Acts as a Prognostic Factor in Lower-Grade Gliomas,” Int. J. Mol. Sci. 17: 1276: 1-11; Li, J. et al. (2016) “Overexpression of ADAM9 Promotes Colon Cancer Cells Invasion,” J. Invest. Surg. 26(3): 127-133). Additionally, ADAM9 and its secreted soluble isoform seem to be crucial for cancer cells to disseminate (Amendola, R. S. et al. (2015) “ADAM9 Disintegrin Domain Activates Human Neutrophils Through An Autocrine Circuit Involving Integrins And CXCR2,” J. Leukocyte Biol. 97(5): 951-962; Fry, J. L. et al. (2010) “Secreted And Membrane-Bound Isoforms Of Protease ADAM9 Have Opposing Effects On Breast Cancer Cell Migration,” Cancer Res. 70, 8187-8198; Mazzocca, A. (2005) “A Secreted Form Of ADAM9 Promotes Carcinoma Invasion Through Tumor-Stromal Interactions,” Cancer Res. 65: 4728-4738; see also U.S. Pat. Nos. 9,150,656; 7,585,634; 7,829,277; 8,101,361; and 8,445,198 and US Patent Publication No. 2009/0023149).

A number of studies have thus identified ADAM9 as a potential target for anticancer therapy (Peduto, L. (2009) “ADAM9 As A Potential Target Molecule In Cancer,” Curr. Pharm. Des. 15: 2282-2287; Duffy, M. J. et al. (2009) “Role Of ADAMs In Cancer Formation And Progression,” Clin. Cancer Res. 15: 1140-1144; Duffy, M. J. et al. (2011) “The ADAMs Family Of Proteases: New Biomarkers And Therapeutic Targets For Cancer?,” Clin. Proteomics 8: 9: 1-13; Josson, S. et al. (2011) “Inhibition of ADAM9 Expression Induces Epithelial Phenotypic Alterations and Sensitizes Human Prostate Cancer Cells to Radiation and Chemotherapy,” Prostate 71(3): 232-240; see also US Patent Publication Nos. 2016/0138113, 2016/0068909, 2016/0024582, 2015/0368352, 2015/0337356, 2015/0337048, 2015/0010575, 2014/0342946, 2012/0077694, 2011/0151536, 2011/0129450, 2010/0291063, 2010/0233079, 2010/0112713, 2009/0285840, 2009/0203051, 2004/0092466, 2003/0091568, and 2002/0068062, and PCT Publication Nos. WO 2016/077505, WO 2014/205293, WO 2014/186364, WO 2014/124326, WO 2014/108480, WO 2013/119960, WO 2013/098797, WO 2013/049704, and WO 2011/100362). Additionally, the expression of ADAM9 has also been found to be relevant to pulmonary disease and inflammation (see, e.g., US Patent Publication Nos. 2016/0068909; 2012/0149595; 2009/0233300; 2006/0270618; and 2009/0142301). An anti-ADAM9-targeting antibody-drug conjugate (ADC), IMGC936, is currently in a Phase I dose escalation study evaluating safety and pharmacokinetics in cancer patients.

However, despite all prior advances, a need remains high for more effective ADAM9 targeting therapeutics and methods for the treatment of cancer.

### SUMMARY OF THE INVENTION

The present invention is based on the discovery of a dynamic range of expression of ADAM9 in tumor tissue and the discovery that tumors with increased levels of ADAM9 expression are more responsive to treatment with anti-ADAM9 immunoconjugates. The present invention advantageously permits treatment of patients who have a greater likelihood of responding to treatment by administering therapeutic agents, i.e., anti-ADAM9 immunoconjugates, to patients who are found to have an increased expression level of ADAM9.

The present invention also provides a method of treating cancer or a method for increasing the likelihood of effectiveness of a cancer treatment, the method comprising administering a therapeutically effective dose of an anti-ADAM9 immunoconjugate, to a subject, wherein ADAM9 expression in a tissue sample from the subject has been found to be elevated.

In one embodiment, the extent and uniformity of ADAM9 expression is detected by immunohistochemistry (IHC). In another embodiment, the level of ADAM9 expression is detected by calibrated IHC. Non-limiting examples of IHC include IHC methods that distinguish between varying levels of ADAM9 and calibrated IHC methods, such as those described herein. The ADAM9 expression can be scored using an appropriate scoring system, including, but not limited to, the scoring methods described herein. For example, ADAM9 expression can be scored using a calibrated IHC method that includes a range of 0, 1, 2, or 3 for staining intensity with 0 being the lowest level of staining intensity and 3 being the highest level of staining intensity. In certain embodiments, a staining intensity score of 2, or 3 is indicative of an elevated ADAM9 expression level. In some embodiments, a staining intensity score of 1 is considered as “weak” staining; a staining intensity score of 2 is considered as “moderate” staining; and a staining intensity score of 3 is considered as “strong” staining.

Alternatively or additionally, ADAM9 expression can be scored using a calibrated IHC method that includes a staining uniformity expressed as percentage of cells with staining intensity of 0, 1, 2, or 3. For example, the ADAM9 expression level in a sample can be scored by combining staining intensity and staining uniformity, e.g., PS1, PS2 or PS3. In certain embodiments, a staining of 25% or greater, 50% or greater or 75% or greater PS1 is indicative of an increase in ADAM9 expression level. In certain embodiments, a staining of 25%-49%, 50%-74% or 75%-100% PS1 is indicative of an increase in ADAM9 expression level. In some embodiments, a staining of 25% or greater, 50% or greater or 75% or greater PS2 is indicative of an increase in ADAM9 expression level. In some embodiments, a staining of 25%-49%, 50%-74% or 75%-100% PS2 is indicative of an increase in ADAM9 expression level. In some embodiments, a staining of 25% or greater, 50% or greater or 75% or greater PS3 is indicative of an increase in ADAM9 expression level. In some embodiments, a staining of 25%-49%, 50%-74% or 75%-100% PS3 is indicative of an increase in ADAM9 expression level.

In some embodiments, the level of ADAM9 expression in a sample is assigned to a Tumor Proportion Score (TPS). In certain embodiments, a tumor sample with a TPS of greater or equal to 1%, greater or equal to 5%, greater or equal to 10%, greater or equal to 20%, greater or equal to 25%, greater or equal to 30%, greater or equal to 40%, greater or equal to 50%, greater or equal to 60%, greater or equal to 70%, greater or equal to 75%, greater or equal to 80%, greater or equal to 90%, or greater or equal to 95% is indicative of an increased ADAM9 expression. In certain embodiments, a tumor sample with a TPS of greater or equal to 25% is indicative of an increased ADAM9 expression. In certain embodiments, a tumor sample with a TPS of greater or equal to 50% is indicative of an increased ADAM9 expression. In certain embodiments, a tumor sample with a TPS of greater or equal to 75% is indicative of an increased ADAM9 expression.

In some embodiments, the level of ADAM9 expression in a sample is assigned to a H-score, which combines components of staining intensity with the percentage of positive cells in the sample. In certain embodiments, a tumor sample with a H-score between 50 and 300 is indicative of an increased ADAM9 expression. In certain embodiments, a tumor sample with an H-score between 100 and 300 is indicative of an increased ADAM9 expression. In certain embodiments, the tumor sample has a high ADAM9 expression level with an H-score between 201 and 300. In certain embodiments, the tumor sample has a medium ADAM9 expression level with a H-score between 101 and 200. In certain embodiments, the tumor sample has a low ADAM9 expression level with an H-score between 1 and 100.

In a further embodiment, the ADAM9 expression in a sample (e.g., a tumor tissue sample) is measured and compared to one or more reference samples. In one embodiment, the ADAM9 expression in the sample is compared to a negative control sample which demonstrates no or low detectable ADAM9 expression. In another embodiment, the ADAM9 expression in the sample is compared to a positive control sample having increased ADAM9 expression (level 1 , 2, or 3).

In certain embodiments, the anti-ADAM9 immunoconjugate that can be used in the methods of the present invention comprises an anti-ADAM9 antibody or an ADAM9 binding fragment thereof, a linker, and a cytotoxin. In one embodiment, the linker can be selected from the group consisting of a cleavable linker, a non-cleavable linker, a hydrophilic linker, and a dicarboxylic acid-based linker. In another embodiment, the linker can be selected from the group consisting: N-succinimidyl 4-(2-pyridyldithio)pentanoate (SPP) or N-succinimidyl 4-(2-pyridyldithio)-2-sulfopentanoate (sulfo-SPP); N-succinimidyl 4-(2-pyridyldithio)butanoate (SPDB) or N-succinimidyl 4-(2-pyridyldithio)-2-sulfobutanoate (sulfo-SPDB); N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(N-maleimidopropionamido)-tetraethyleneglycol] ester (NHS -PEG4-maleimide). In another embodiment, the linker is N-succinimidyl 4-(2-pyridyldithio)-2-sulfobutanoate (sulfo-SPDB). In another embodiment, the cytotoxic agent is selected from the group consisting of a maytansinoid, maytansinoid analog, benzodiazepine, taxoid, CC-1065, CC-1065 analog, duocarmycin, duocarmycin analog, calicheamicin, dolastatin, dolastatin analog, auristatin, tomaymycin derivative, and leptomycin derivative or a prodrug of the agent. In another embodiment, the cytotoxic agent is a maytansinoid. In another embodiment, the cytotoxic agent is N(2′)-deacetyl-N(2′)-(3-mercapto-1 -oxopropyl)-maytansine or N(2′)-deacetyl-N2-(4-mercapto-4-methyl-1 -oxopentyl)-maytansine. In another embodiment, the cytotoxic agent is N(2′)-deacetyl-N2-(4-mercapto-4-methyl-1-oxopentyl)-maytansine (DM4).

In certain embodiments, the anti-ADAMS immunoconjugate is represented by the following formula:

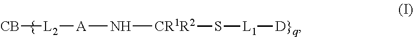

or a pharmaceutically acceptable salt thereof, wherein:

CB is an anti-ADAMS antibody or ADAMS-binding fragment thereof;

L₂ is represented by one of the following formula:

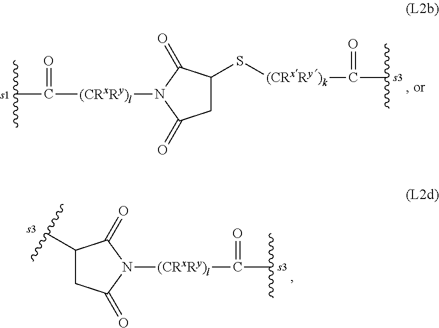

wherein:
R^{x}, R^{y}, R^{x′} and R^{y′}, for each occurrence, are independently H, —OH, halogen, —O-(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;

L₁ is represented by the following formula:

—CR³R⁴—(CH₂)₁₋₈—C(═O)—

wherein R³ and R⁴ are each independently H or Me, and the —C(═O)— moiety in L₁ is connected to D;

D is represented by the following formula:

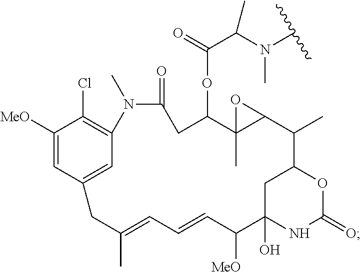 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

q is an integer from 1 to 20.

In certain embodiments, the anti-ADAMS immunoconjugate of the present invention

is represented by the following formula:

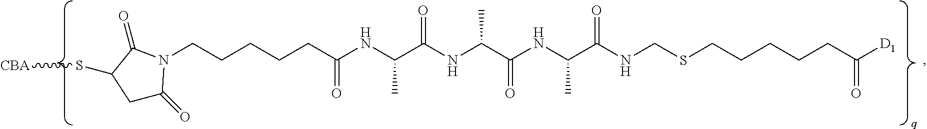

wherein:
CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
q is 1 or 2;
D₁ is represented by the following formula:

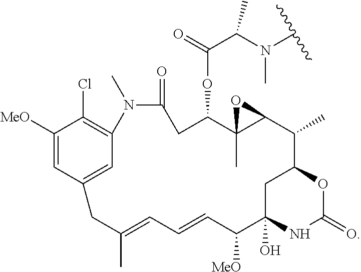 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the humanized anti-ADAM9 antibody or ADAM9-binding fragment thereof comprises a heavy chain variable domain (VH) and a light chain variable domain (VL) having sequences of SEQ ID NO:33 and SEQ ID NO:35, respectively. In some embodiments, the humanized anti-ADAM9 antibody comprises a heavy chain and a light chain having the sequences of SEQ ID NO:42 and SEQ ID NO:50, respectively. In some embodiments, the humanized anti-ADAM9 antibody comprises a heavy chain and a light chain having the sequences of SEQ ID NO:45 and SEQ ID NO:50, respectively. In some embodiments, In some embodiments, X in SEQ ID NO:42 or SEQ ID NO:45 is lysine. In some embodiments, In some embodiments, X in SEQ ID NO:42 or SEQ ID NO:45 is absent. In some embodiments, the humanized anti-ADAM9 antibody comprises a heavy chain and a light chain having the sequences of SEQ ID NO:49 and SEQ ID NO:50, respectively. In some embodiments, the DAR value for a composition (e.g., pharmaceutical compositions) comprising the immunoconjugate is in the range of 1.0 to 2.5, 1.5 to 2.5, 1.8 to 2.2, or 1.9 to 2.1. In some embodiments, the DAR is 1.8, 1.9, 2.0 or 2.1.

Also provided in the present invention is the use of the immunoconjugate or the pharmaceutical composition of the present invention described herein in a method of treating cancer in a subject in need of the treatment or a method of increasing the efficacy of cancer treatment in a subject in need of the treatment, wherein a tumor sample from the subject exhibits an increased level of ADAM9 expression. The present invention also provides the use of the immunoconjugate or the pharmaceutical composition of the present invention described herein for the manufacture of a medicament for treating cancer in a subject in need of the treatment or for increasing the efficacy of cancer treatment in a subject in need of the treatment, wherein a tumor sample from the subject exhibits an increased level of ADAM9 expression.

In certain embodiments, the cancer is selected from the group consisting of lung cancer, colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer. In certain embodiments, the cancer is non-small-cell lung cancer (NSCLC), colorectal cancer, gastric cancer, breast cancer, or pancreatic cancer. In certain embodiments, the cancer is adenocarcinoma NSCLC, triple negative breast cancer (TNBC), pancreatic cancer, gastric cancer or colorectal cancer (CRC).

### BRIEF DESCRIPTION OF THE DRAWINGS

FIG. 1A is a graph showing ADAM9 expression and response in PDX models of various cancers. FIG. 1B shows ADAM9 prevalence data in each cancer type. FIG. 1C shows ADAM9 staining intensity and H-score for each PDX model of triple negative breast cancer (TNBC), pancreatic cancer, gastric cancer and adenocarcinoma non-small cell lung cancer (NSCLC).

FIG. 2 shows ADAM9 prevalence data in gastric cancer, pancreatic cancer, adenocarcinoma non-small cell lung cancer (NSCLC), triple negative breast cancer (TNBC), colorectal cancer (CRC), and esophageal cancer using the internal research ADAM9 assay.

FIG. 3 shows ADAM9 prevalence data in gastric cancer, pancreatic cancer, adenocarcinoma non-small cell lung cancer (NSCLC), triple negative breast cancer (TNBC), colorectal cancer (CRC), and esophageal cancer using the Roche Tissue Diagnostics (RTD) ADAM9 robust prototype assay (RPA).

FIGS. 4A-4C are graphs showing ADAM9 expression in gastric cancer tissue samples. FIG. 4A is shows ADAM9 H-score for positive membrane and cytoplasmic staining. FIG. 4B shows ADAM9 tumor proportion score for positive membrane and cytoplasmic staining. FIG. 4C shows ADAM9 tumor proportion score for positive membrane staining only.

FIGS. 5A-5C are graphs showing ADAM9 expression in pancreatic cancer tissue samples. FIG. 5A shows ADAM9 H-score for positive membrane and cytoplasmic staining. FIG. 5B shows ADAM9 tumor proportion score for positive membrane and cytoplasmic staining. FIG. 5C shows ADAM9 tumor proportion score for positive membrane staining only.

FIGS. 6A-6C are graphs showing ADAM9 expression in adenocarcinoma NSCLC tissue samples. FIG. 6A shows H-score for positive membrane and cytoplasmic staining. FIG. 6B shows ADAM9 tumor proportion score for positive membrane and cytoplasmic staining. FIG. 6C shows ADAM9 tumor proportion score for positive membrane staining only.

FIGS. 7A-7C are graphs showing ADAM9 expression in TNBC tissue samples. FIG. 7A shows H-score for positive membrane and cytoplasmic staining. FIG. 7B shows ADAM9 tumor proportion score for positive membrane and cytoplasmic staining. FIG. 7C shows ADAM9 tumor proportion score for positive membrane staining only.

FIGS. 8A-8C are graphs showing ADAM9 expression in CRC tissue samples. FIG. 8A shows H-score for positive membrane and cytoplasmic staining. FIG. 8B shows ADAM9 tumor proportion score for positive membrane and cytoplasmic staining. FIG. 8C shows ADAM9 tumor proportion score for positive membrane staining only.

FIGS. 9A-9C are graphs showing ADAM9 expression in esophageal cancer tissue samples. FIG. 9A shows H-score for positive membrane and cytoplasmic staining. FIG. 9B shows ADAM9 tumor proportion score for positive membrane and cytoplasmic staining. FIG. 9C shows ADAM9 tumor proportion score for positive membrane staining only.

### DETAILED DESCRIPTION OF THE INVENTION

The present disclosure provides methods of increasing the efficacy of or likelihood of response to the treatment of cancers characterized by the overexpression of ADAM9. The present disclosure is based on the discovery of a dynamic range of expression of ADAM9 in tumor tissues as compared to normal tissue and the discovery that tumors with increased levels of ADAM9 expression are more responsive to treatment of anti-ADAM9 immunoconjugates.

Patients with a cancer that is likely to respond to an anti-ADAM9 immunoconjugate can be identified by: (a) contacting a biological sample comprising cells from said cancer with an agent that binds ADAM9 protein of the biological sample (e.g., on the cell surface and/or inside the cell); (b) detecting binding of said agent that binds ADAM9 protein of said biological sample of (a); (c) assigning a score to said binding of step (b), wherein said score is assigned based on comparison to one or more reference samples; and (d) comparing said score in step (c) to the score of a reference tissue or cell, wherein a score for said cancer ADAM9 level that is greater than the score for a normal or low ADAM9 expressing reference sample or a score for said cancer ADAM9 level that is equal to or greater than the score for a high ADAM9 expressing reference sample identifies said cancer as likely to respond to an anti-ADAM9 immunoconjugate.

A tumor that is sensitive to treatment with an anti-ADAM9 immunoconjugate can be identified by: (a) measuring the level of ADAM9 expression in a tumor tissue sample obtained from said tumor, wherein said measuring comprises the use of a detection method that distinguishes between staining intensity or staining uniformity in a ADAM9 expressing cancer sample as compared to staining intensity or staining uniformity in one or more reference samples; (b) determining a ADAM9 staining intensity score for said tumor tissue sample; and (c) comparing the ADAM9 staining intensity score determined in step (b) to a relative value determined by measuring ADAM9 protein expression in at least one reference sample, wherein said at least one reference sample is a tissue, cell, or cell pellet sample which is not sensitive to treatment with anti-ADAM9 immunoconjugate, and wherein a ADAM9 staining intensity score for said sample determined in step (b) that is higher than said relative value identifies said tumor as being sensitive to treatment with an anti-ADAM9 immunoconjugate. In certain embodiments, the detection method is performed manually or using an automated system. In one embodiment, the detection method is IHC. In another embodiment, the IHC is calibrated IHC that can distinguish different levels of ADAM9 expression.

### I. Definitions

The terms “ADAM9 ” or “Disintegrin and Metalloproteinase Domain-containing Protein 9” as used herein, refers to any native human ADAM9, unless otherwise indicated. The term “ADAM9 ” encompasses “full- length,” unprocessed ADAM (as well as any form of ADAM9 that results from processing within the cell). The term also encompasses naturally occurring variants of ADAM9, e.g., splice variants, allelic variants and isoforms. The ADAM9 polypeptides described herein can be isolated from a variety of sources, such as from human tissue types or from another source, or prepared by recombinant or synthetic methods. Examples of ADAM9 sequences include, but are not limited to NCBI reference numbers NP_003807, and those described in WO2018/119166, WO2018/119196 and WO2020/005945, which are herein incorporated by reference.

The terms “overexpression”, “increased expression” or “elevated expression” are used interchangeably. An increased expression of ADAM9 refers to a sample which contains elevated levels of ADAM9 expression. In one example, the ADAM9 expression is measured by IHC and given a staining intensity score and/or a staining uniformity score by comparison to controls (e.g., calibrated controls) exhibiting defined scores (e.g. an intensity score of 3 is given to the test sample if the intensity is comparable to the level 3 calibrated control or an intensity of 2 is given to the test sample if the intensity is comparable to the level 2 calibrated control). In some embodiments, the staining intensity score is determined by a pathologist based on his/her pathology training and experience without a calibrated control. For example, a score of 1, 2, or 3 by immunohistochemistry indicates an increased expression of ADAM9. In certain embodiments, a score of 2 or 3 is indicative of an increased expression of ADAM9. In some embodiments, the presence of cells in the sample that have staining intensity of 3 is indicative of increased ADAM9 expression. Staining uniformity is also indicative of increased ADAM9 expression. A staining uniformity score can be the percentage of cells in the sample with the same staining intensities.

The staining intensity and staining uniformity scores can be used alone or in combination (e.g., PS1, PS2, PS3, etc.). PS1 (also known as PS1+) represents the percentage of tumor cells in the sample with staining intensity of 1 or greater (e.g., 2, or 3). PS2 (also known as PS2+) represents the percentage of tumor cells in the sample with staining intensity of 2 or greater (e.g., 3). PS3 (also known as PS3+) represents the percentage of tumor cells in the sample with staining intensity of 3. In some embodiments, a staining of 1%-24%, 25%-49%, 50%-74% or 75%-100% PS1 is indicative of increased ADAM9 expression. In some embodiments, a staining of 1% or greater, 5% or greater, 10% or greater, 20% or greater, 25% or greater, 30% or greater, 40% or greater, 50% or greater, 60% or greater, 70% or greater, 75% or greater, 80% or greater, 90% or greater, or 95% or greater PS1 is indicative of increased ADAM9 expression. In some embodiments, a staining of 25% or greater PS1 is indicative of increased ADAM9 expression. In some embodiments, a staining of 50% or greater PS1 is indicative of increased ADAM9 expression. In some embodiments, a staining of 75% or greater PS1 is indicative of increased ADAM9 expression. In some embodiments, a staining of 1%-24%, 25%-49%, 50%-74% or 75%-100% PS2 is indicative of increased ADAM9 expression. In some embodiments, a staining of 1% or greater, 5% or greater, 10% or greater, 20% or greater, 25% or greater, 30% or greater, 40% or greater, 50% or greater, 60% or greater, 70% or greater, 75% or greater, 80% or greater, 90% or greater, or 95% or greater PS2 is indicative of increased ADAM9 expression. In some embodiments, a staining of 25% or greater PS2 is indicative of increased ADAM9 expression. In some embodiments, a staining of 50% or greater PS2 is indicative of increased ADAM9 expression. In some embodiments, a staining of 75% or greater PS2 is indicative of increased ADAM9 expression. In some embodiments, a staining of 1%-24%, 25%-49%, 50%-74% or 75%-100% PS3 is indicative of increased ADAM9 expression. In some embodiments, a staining of 1% or greater, 5% or greater, 10% or greater, 20% or greater, 25% or greater, 30% or greater, 40% or greater, 50% or greater, 60% or greater, 70% or greater, 75% or greater, 80% or greater, 90% or greater, or 95% or greater PS3 is indicative of increased ADAM9 expression. In some embodiments, a staining of 25% or greater PS3 is indicative of increased ADAM9 expression. In some embodiments, a staining of 50% or greater PS3 is indicative of increased ADAM9 expression. In some embodiments, a staining of 75% or greater PS3 is indicative of increased ADAM9 expression. In some embodiments, a staining of 50% or greater PS1 and 25% or greater PS2 is indicative of increased ADAM9 expression. In some embodiments, a staining of 75% or greater PS1 and 25% or greater PS2 is indicative of increased ADAM9 expression. In some embodiments, a staining of 50% or greater PS1 and 50% or greater PS2 is indicative of increased ADAM9 expression. In some embodiments, a staining of 75% or greater PS1 and 50% or greater PS2 is indicative of increased ADAM9 expression. In some embodiments, a staining of 75% or greater PS1 and 75% or greater PS2 is indicative of increased ADAM9 expression. In some embodiments, a sample having a staining of greater than or equal to 50% PS1 is considered as “ADAM9 positive”. Methods described herein can be used in treating a subject whose tumor sample is ADAM9 positive by administering to the subject a therapeutically effective amount of an anti-ADAM9 immunoconjugate described herein.

In some embodiments, Tumor Proportion Score (TPS) can be used to indicate positive ADAM9 expression or “increased ADAM expression”. TPS is calculated as percentage of viable tumor cells in the sample staining at any intensity. In some embodiments, a TPS is for membrane and cytoplasmic staining. In some embodiments, a TPS is for membrane staining only. For membrane staining, a TPS includes both complete or partial staining. As use herein, “complete” membrane staining means staining around the whole membrane and “partial” membrane staining means apical staining of the membrane. In some embodiments, a TPS of greater or equal to 1%, greater or equal to 5%, greater or equal to 10%, greater or equal to 20%, greater or equal to 25%, greater or equal to 30%, greater or equal to 40%, greater or equal to 50%, greater or equal to 60%, greater or equal to 70%, greater or equal to 75%, greater or equal to 80%, greater or equal to 90%, or greater or equal to 95% is considered as “ADAM positive” or “increased ADAM9 expression”. In some embodiments, a TPS of greater or equal to 25% is considered as “ADAM positive” or “increased ADAM9 expression”. In some embodiments, a TPS of greater or equal to 50% is considered as “ADAM positive” or “increased ADAM9 expression”. In some embodiments, a TPS of greater or equal to 75% is considered as “ADAM positive” or “increased ADAM9 expression”

In another example, the IHC staining results are analyzed by H-score, which combines components of staining intensity with the percentage of positive cells. It has a value between 0 and 300 and is defined as:

1*(percentage of cells staining at intensity category 1)+2*(percentage of cells staining at intensity category 2)+3*(percentage of cells staining at intensity category 3)=H-score.

In some embodiments, a tumor with an elevated ADAM9 expression has a higher H-score when compared to an H-score of a reference sample, for example, from a subject without cancer or with a cancer that does not have elevated ADAM9 expression).

In some embodiments, the ADAM9 expression level in the patient sample is high. In some embodiments, the ADAM9 expression level in the patient sample is medium. In some embodiments, the ADAM9 expression level in the patient sample is low. As used herein, a sample with an H-score of 201-300 is considered as having “high” ADAM9 expression level. A sample with an H-score of 101-200 is considered as having “medium” ADAM9 expression level. A sample with an H-score of 1-100 is considered as having “low” ADAM9 expression level.

In another example, an increase in ADAM9 expression can be determined by detection of an increase of at least 2-fold, at least 3-fold, or at least 5-fold relative to control values (e.g., expression level in a tissue or cell from a subject without cancer or with a cancer that does not have elevated ADAM9 values).

In some embodiments, the staining intensity, staining uniformity and staining scores described above are for membrane staining only. In some embodiments, the staining intensity, staining uniformity and staining scores described above are for cytoplasmic staining. In some embodiments, the staining intensity, staining uniformity and staining scores described above are for the combination of membrane staining and cytoplasmic staining (cyto-membrane staining).

A “reference sample” can be used to correlate and compare the results obtained in the methods of the present invention from a test sample. Reference samples can be cells (e.g., cell lines, cell pellets) or tissue. The ADAM9 levels in the “reference sample” may be an absolute or relative amount, a range of amounts, a minimum and/or maximum amount, a mean amount, and/or a median amount of ADAM9. The diagnostic methods of the disclosure involve a comparison between expression levels of ADAM9 in a test sample and a “reference value.” In some embodiments, the reference value is the expression level of the ADAM9 in a reference sample. A reference value may be a predetermined value and may also be determined from reference samples (e.g., control biological samples or determined by a pathologist based on his/her pathology training and experience without a control biological sample) tested in parallel with the test samples. A reference value can be a single cut-off value, such as a median or mean or a range of values, such as a confidence interval. Reference values can be established for various subgroups of individuals, such as individuals predisposed to cancer, individuals having early or late stage cancer, male and/or female individuals, or individuals undergoing cancer therapy. Examples of normal reference samples or values and positive reference samples or values are described herein.

In some embodiments, the reference sample is a sample from a healthy tissue, in particular a corresponding tissue which is not affected by cancer. These types of reference samples are referred to as negative control samples. In other embodiments, the reference sample is a sample from a tumor tissue that expresses ADAM9. These types of reference samples are referred to as positive control samples. Positive control samples can also be used as a comparative indicator for the uniformity (cell percentage for cells with the same staining intensity) and/or degree (1, 2, 3) of staining intensity, which correlates with the level of ADAM9 expression. Positive control comparative samples are also referred to as calibrated reference samples which demonstrate a dynamic range of staining intensity or uniformity. Appropriate positive and negative reference levels of ADAM9 for a particular cancer, may be determined by measuring levels of ADAM9 in one or more appropriate subjects, and such reference levels may be tailored to specific populations of subjects (e.g., a reference level may be age-matched so that comparisons may be made between ADAM9 levels in samples from subjects of a certain age and reference levels for a particular disease state, phenotype, or lack thereof in a certain age group). Such reference levels may also be tailored to specific techniques that are used to measure levels of ADAM9 in biological samples (e.g., immunoassays, etc.), where the levels of ADAM9 may differ based on the specific technique that is used.

The term “primary antibody” herein refers to an antibody that binds specifically to the target protein antigen in a tissue sample. A primary antibody is generally the first antibody used in an immunohistochemical (IHC) procedure. In one embodiment, the primary antibody is the only antibody used in an IHC procedure. The term “secondary antibody” herein refers to an antibody that binds specifically to a primary antibody, thereby forming a bridge between the primary antibody and a subsequent reagent, if any. The secondary antibody is generally the second antibody used in an immunohistochemical procedure.

A “sample” or “biological sample” of the present invention is of biological origin, in specific embodiments, such as from eukaryotic organisms. In preferred embodiments, the sample is a human sample, but animal samples may also be used in the practice of the invention. The present invention is particularly useful for cancer samples which generally comprise solid tissue samples. The method can be used to examine an aspect of expression of ADAM9 or a state of a sample, including, but not limited to, comparing different types of cells or tissues, comparing different developmental stages, and detecting or determining the presence and/or type of disease or abnormality.

For the purposes herein, a “section” of a tissue sample refers to a single part or piece of a tissue sample, e.g. a thin slice of tissue or cells cut from a tissue sample. It is understood that multiple sections of tissue samples may be taken and subjected to analysis according to the present invention. In some cases, the selected portion or section of tissue comprises a homogeneous population of cells. In other cases, the selected portion comprises a region of tissue, e.g. the lumen as a non-limiting example. The selected portion can be as small as one cell or two cells, or could represent many thousands of cells, for example.

The term “correlate” or “correlating” means comparing, in any way, the performance and/or results of a first analysis with the performance and/or results of a second analysis. For example, one may use the results of a first analysis in carrying out the second analysis and/or one may use the results of a first analysis to determine whether a second analysis should be performed and/or one may compare the results of a first analysis with the results of a second analysis. In one embodiment, increased expression of ADAM9 correlates with increased likelihood of effectiveness of a ADAM9-targeting anti-cancer therapy.

The term “antibody” means an immunoglobulin molecule capable of specific binding to a target, such as a carbohydrate, polynucleotide, lipid, polypeptide, etc., through at least one antigen recognition site, located in the Variable Domain of the immunoglobulin molecule. As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab') fragments, intrabodies, and epitope-binding fragments of any of the above. In particular, the term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies can be naked or conjugated to other molecules such as toxins, radioisotopes, etc.

Antibodies are capable of “immunospecifically binding” to a polypeptide or protein or a non-protein molecule due to the presence on such molecule of a particular domain or moiety or conformation (an “epitope”). An epitope-containing molecule may have immunogenic activity, such that it elicits an antibody production response in an animal; such molecules are termed “antigens.” As used herein, an antibody is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds that viral epitope with greater affinity, avidity, more readily, and/or with greater duration than it immunospecifically binds to other viral epitopes or to non-viral epitopes. It is also understood by reading this definition that, for example, an antibody (or moiety or epitope) that immunospecifically binds to a first target may or may not specifically or preferentially bind to a second target. As such, “immunospecific binding” to a particular epitope does not necessarily require (although it can include) exclusive binding to that epitope. Generally, but not necessarily, reference to binding means “immunospecific” binding. Two molecules are said to be capable of binding to one another in a “physiospecific” manner, if such binding exhibits the specificity with which receptors bind to their respective ligands.

The term “monoclonal antibody” refers to a homogeneous antibody population wherein the monoclonal antibody is comprised of amino acids (naturally occurring or non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. The term is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256: 495-497, or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3): 119-125). In general, cells should be kept intact and preferably viable when used as immunogens. Intact cells may allow antigens to be better detected than ruptured cells by the immunized animal. Use of denaturing or harsh adjuvants, e.g., Freund's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate an affinity optimized, a chimeric antibody, a humanized antibody, and/or a caninized antibody, to improve the affinity, or other characteristics of the antibody, as well as the immunoconjugates of the invention. The general principle in humanizing an antibody involves retaining the basic sequence of the antigen-binding portion of the antibody, while swapping the non-human remainder of the antibody with human antibody sequences.

Natural antibodies (such as natural IgG antibodies) are composed of two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. In contrast, scFvs are single chain molecules made by linking Light and Heavy Chain Variable Domains together via a short linking peptide.

The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Region. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide).

The term “anti-ADAM9 antibody” or “an antibody that binds to ADAM9” refers to an antibody that is capable of binding ADAM9 with sufficient affinity such that the antibody is useful as a diagnostic and/or therapeutic agent in targeting ADAM9. The extent of binding of an anti-ADAM9 antibody to an unrelated, non-ADAM9 protein is less than about 10% of the binding of the antibody to ADAM9 as measured, e.g., by a radioimmunoassay (RIA). In certain embodiments, an antibody that binds to ADAM9 has a dissociation constant (Kd) of ≤1 μM, ≤100 nM, ≤10 nM, ≤1 nM, or ≤0.1 nM. Examples of anti-ADAMS antibodies are known in the art and are disclosed in WO2018/119166, WO2018/119196 and WO2020/005945, which are herein incorporated by reference. Other exemplary antibodies that bind to ADAMS are commercially available from Abcam, Thermofisher, Sigma-Aldrich, and other companies. Examples of commercially available anti-ADAMS antibodies include anti-ADAMS antibodies from the following companies: Cell Signaling Technology Product #2099 and #4151, ThermoFisher Product #PA5-14338, #PA5-17080, and #A5-76732, Abcam ab186833, Sigma-Aldrich Product #HPA004000, GeneTex Catalog No. GTX130025, LifeSpan BioSciences LS-C100638, LS-0502670 and LS-C124856, Novus Biologicals Catalog #27120002, Affinity Biosciences Catalog #AF7559, CusaBio CSB-PA618774ESR2HU, ProSci Catalog #19-633 and 62-909, Biorbyt Catalog #orb229445 and orb192735, Bioss Catalog #bs-4204R and bs-4304R-Biotin, G Biosciences Catalog #ITA7379, Abbex Catalog #abx103793, and Spring Bioscience anti-ADAM9 antibody (J12H2L3).

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

The term “immunoconjugate” or “conjugate” as used herein refers to a compound or a derivative thereof that is linked to a cell binding agent (i.e., an anti-ADAMS antibody or fragment thereof) and is defined by a generic formula: C-L-A, wherein C=cytotoxin, L=linker, and A=cell binding agent or anti-ADAMS antibody or antibody fragment. Immunoconjugates can also be defined by the generic formula in reverse order: A-L-C.

A “linker” is any chemical moiety that is capable of linking a compound, usually a drug, such as a maytansinoid, to a cell-binding agent such as an anti-ADAMS antibody or a fragment thereof in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. In some embodiments, examples of cancer include non-small-cell lung cancer (NSCLC), colorectal cancer, gastric cancer, breast cancer, or pancreatic cancer. In some embodiments, examples of cancer include adenocarcinoma NSCLC, triple negative breast cancer (TNBC), pancreatic cancer, gastric cancer or colorectal cancer.

“Tumor” and “neoplasm” refer to any mass of tissue that result from excessive cell growth or proliferation, either benign (noncancerous) or malignant (cancerous) including pre-cancerous lesions.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical formulation” refers to a preparation which is in such form as to permit the biological activity of the active ingredient to be effective, and which contains no additional components which are unacceptably toxic to a subject to which the formulation would be administered. Such formulation can be sterile.

An “effective amount” of an antibody or an antibody immunoconjugate as disclosed herein is an amount sufficient to carry out a specifically stated purpose.

The term “therapeutically effective amount” refers to an amount of an antibody, an antibody immunoconjugate or other drug effective to “treat” a disease or disorder in a subject or mammal. In the case of cancer, the therapeutically effective amount of the drug can reduce the number of cancer cells; reduce the tumor size; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; and/or relieve to some extent one or more of the symptoms associated with the cancer. See the definition herein of “treating”. To the extent the drug can prevent growth and/or kill existing cancer cells, it can be cytostatic and/or cytotoxic.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50: 1 S-10S (2009)). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C<42% is the minimum level of anti-tumor activity. A T/C<10% is considered a high anti-tumor activity level, with T/C (%)=Median tumor volume of the treated/Median tumor volume of the control×100. Other favorable responses to the cancer treatment can include increased progression-free survival (PFS), disease-free survival (DFS), or overall survival (OS), complete response (CR), partial response (PR), or, in some cases, stable disease (SD), a decrease in progressive disease (PD), a reduced time to progression (TTP).

The word “label” when used herein refers to a detectable compound or composition which is conjugated directly or indirectly to the antibody so as to generate a “labeled” antibody. The label can be detectable by itself (e.g. radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented, in certain embodiments, a subject is successfully “treated” for cancer according to the methods of the present invention if the patient shows one or more of the following: reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival (PFS), overall survival (OS), each as measured by standards set by the National Cancer Institute and the U.S. Food and Drug Administration for the approval of new drags. See Johnson et al, (2003) J. Clin. Oncol. 21(7): 1404-1411.

“Progression free survival” (PFS), also referred to as or “Time to Tumor Progression” (YIP) indicates the length of time during and after treatment that the cancer does not grow. Progression-free survival includes the amount of time patients have experienced a complete response or a partial response, as well as the amount of time patients have experienced stable disease.

A “complete response” or “complete remission” or “CR” indicates the disappearance of all signs of tumor or cancer in response to treatment. This does not always mean the cancer has been cured.

A “partial response” or “PR” refers to a decrease in the size or volume of one or more tumors or lesions, or in the extent of cancer in the body, in response to treatment.

“Stable disease” refers to disease without progression or relapse. In stable disease there is neither sufficient tumor shrinkage to qualify for partial response nor sufficient tumor increase to qualify as progressive disease.

“Progressive disease” refers to the appearance of one more new lesions or tumors and/or the unequivocal progression of existing non-target lesions. Progressive disease can also refer to a tumor growth of more than 20 percent since treatment began, either due to an increases in mass or in spread of the tumor.

“Disease free survival” (DFS) refers to the length of time during and after treatment that the patient remains free of disease.

“Overall Survival” (OS) refers to a prolongation in life expectancy as compared to naive or untreated individuals or patients.

“Alkyl” as used herein refers to a saturated linear or branched-chain monovalent hydrocarbon radical of one to twenty carbon atoms. Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Preferably, the alkyl has one to ten carbon atoms. More preferably, the alkyl has one to four carbon atoms.

The number of carbon atoms in a group can be specified herein by the prefix “Cₓ₋ₓₓ”, wherein x and xx are integers. For example, “C₁₋₄alkyl” is an alkyl group having from 1 to 4 carbon atoms.

The term “compound” or “cytotoxic compound,” or “cytotoxic agent” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, and salts (e.g., pharmaceutically acceptable salts) of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt”, “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

“Diastereomer” refers to a stereoisomer with two or more centers of chirality and whose molecules are not mirror images of one another. Diastereomer have different physical properties, e.g. melting points, boiling points, spectral properties, and reactivities. Mixtures of diastereomer can separate under high resolution analytical procedures such as crystallization, electrophoresis and chromatography.

“Enantiomers” refer to two stereoisomers of a compound that are non-superimposable mirror images of one another.

Stereochemical definitions and conventions used herein generally follow S. P. Parker, Ed., McGraw-Hill, Dictionary of Chemical Terms (1984) McGraw-Hill Book Company, New York; and Eliel, E. and Wilen, S., Stereochemistry of Organic Compounds, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “cation” refers to an ion with positive charge. The cation can be monovalent (e.g., Na⁺, K⁺, NH₄⁺ etc.), bi-valent (e.g., Ca²⁺, Mg²⁺, etc.) or multi-valent (e.g., Al³⁺ etc.). Preferably, the cation is monovalent

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′ -methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt can involve the inclusion of another molecule such as an acetate ion, a succinate ion or other counter ion. The counter ion can be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The term “amino acid” refers to naturally occurring amino acids or non-naturally occurring amino acid. In one embodiment, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heterocycyclic ring (e.g., as in proline). The term “amino acid residue” refers to the corresponding residue when one hydrogen atom is removed from the amine end of the amino acid and/or the hydroxyl group is removed from the carboxy end of the amino acid, such as —NH—C(R^{aa′}R^{aa})—C(═O)—. When an amino acid or an amino acid residue is referenced without indicating the specific stererochemistry of the alpha carbon, it is meant to include both the L- and R-isomers. For example, “Ala” includes both L-alanine and R-alanine.

The term “peptide” refers to short chains of amino acid monomers linked by peptide (amide) bonds. In some embodiments, the peptides contain 2 to 20 amino acid residues. In other embodiments, the peptides contain 2 to 10 amino acid residus. In yet other embodiments, the peptides contain 2 to 5 amino acid residues. As used herein, when a peptide is a portion of a cytotoxic agent or a linker described herein represented by a specific sequence of amino acids, the peptide can be connected to the rest of the cytotoxic agent or the linker in both directions. For example, a dipeptide X1-X2 includes X1-X2 and X2-X1. Similarly, a tripeptide X1-X2-X3 includes X1-X2-X3 and X3-X2-X1 and a tetrapeptide X1-X2-X3-X4 includes X1-X2-X3-X4 and X4-X2-X3-X1. X1, X2, X3 and X4 represents an amino acid residue. When a peptide or a peptide residue is referenced without indicating the stereochemistry of each amino acid or amino acid redidue, it meant to include both L- and R-isomers. However, when the stereochemistry of one or more amino acid or amino acid residue in the peptide or peptide residue is specified as D-isomer, the other amino acid or aminod acid residue in the peptide or peptide residue without specified stereochemistry is meant to include only the natural L-isomer. For example, “Ala-Ala-Ala” meant to include peptides or peptide residues, in which each of the Ala can be either L- or R-isomer; while “Ala-D-Ala-Ala” meant to include L-Ala-D-Ala-L-Ala.

As used herein, Drug-Antibody Ratio (DAR) is the average number of the cytotoxic agent covalently linked to one antibody molecule (i.e., average value of q).

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of and/or “consisting essentially of are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Biological Samples

Biological samples are often fixed with a fixative. Aldehyde fixatives such as formalin (formaldehyde) and glutaraldehyde are typically used. Tissue samples fixed using other fixation techniques such as alcohol immersion (Battifora and Kopinski, J. Histochem. Cytochem, (1986) 34: 1095) are also suitable. The samples used may also be embedded in paraffin. In one embodiment, the tissue samples are both formalin-fixed and paraffin-embedded (FFPE). in another embodiment, the FFPE block is hematoxylin and eosin stained prior to selecting one or more portions for analysis in order to select specific area(s) for the FFPE core sample. Methods of preparing tissue blocks from these particulate specimens have been used in previous IHC studies of various prognostic factors, and/or is well known to those of skill in the art. (see, for example, Abbondanzo et al. Am J Clin Pathol, 1.990 May; 93(5): 698-702; Aflred et al, Arch Surg. 1990 January; 125(1): 107-13).

Briefly, any intact organ or tissue may be cut into fairly small pieces and incubated in various fixatives (e.g. formalin, alcohol, etc.) for varying periods of time until the tissue is “fixed”. The samples may be virtually any intact tissue surgically removed from the body. The samples may be cut into reasonably small piece(s) that fit on the equipment routinely used in histopathology laboratories. The size of the cut pieces typically ranges from a few millimeters to a few centimeters.

### III. Detection Antibody Conjugates

In some embodiments, antibodies against ADAM9, generally of the monoclonal type, are linked to at least one agent to form a detection antibody conjugate. In order to increase the efficacy of antibody molecules as diagnostic it is conventional to link or covalently bind or complex at least one desired molecule or moiety. Such a molecule or moiety may be, but is not limited to, at least one reporter molecule. A reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles and/or ligands, such as biotin.

Any cell binding agent (e.g., an antibody or polypeptide) of sufficient selectivity, specificity or affinity may be employed as the basis for detection of the ADAM9 polypeptide. Such properties may be evaluated using conventional immunological screening methodology known to those of skill in the art. Sites for binding to biological active molecules in the antibody molecule, in addition to the canonical antigen binding sites, include sites that reside in the variable domain that can bind the antigen. In addition, the variable domain is involved in antibody self-binding (Kang et al., 1988) and contains epitopes (idiotopes) recognized by anti-antibodies (Kohler et al., 1989).

Certain examples of protein binding (e.g., antibody) conjugates are those conjugates in which the protein binding agent (e.g., antibody) is linked to a detectable label. “Detectable labels” are compounds and/or elements that can be detected due to their specific functional properties, and/or chemical characteristics, the use of which allows the antibody to which they are attached to be detected, and/or further quantified if desired.

Many appropriate imaging agents are known in the art, as are methods for their attachment to antibodies (see, for e.g., U.S. Pat. Nos. 5,021 ,236; 4,938,948; and 4,472,509, each incorporated herein by reference). The imaging moieties used can be paramagnetic ions; radioactive isotopes; fluorochromes; NMR-detectable substances; and/or X-ray imaging, for example.

Exemplary fluorescent labels contemplated for use as protein binding (e.g., antibody) conjugates include Alexa 350, Alexa 430, Alexa 488, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Dylight 488, Fluorescein Isothiocyanate, Green fluorescent protein (GFP), HEX, 6-JOE, Oregon Green 488, Oregon Green 500, Oregon Green 514, Pacific Blue, Phycoerythrin, REG, Rhodamine Green, Rhodamine Red, tetramethyl rhodamine (TMR), Renographin, ROX, TAMRA, TET, Tetramethylrhodamine, Texas Red, and derivatives of these labels (i.e halogenated analogues, modified with isothiocyanate or other linker for conjugating, etc), for example. An exemplary radiolabel is tritium.

Protein binding (e.g., antibody) detection conjugates can be used in the present invention include those for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase and/or glucose oxidase. Preferred secondary binding ligands are biotin and/or avidin and streptavidin compounds. The use of such labels is well known to those of skill in the art and are described, for example, in U.S. Pat Nos. 3,817,837; 3,850,752; 3,939,350; 3,996,345; 4,277,437; 4,275,149 and 4,366,241 ; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al, 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTP A); ethylenetriaminetetraacetic acid; N- chloro-p-toluenesulfonamide; and/or tetrachloro-3a-6a-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Protein binding (e.g., antibody) conjugates with fluorescein markers are prepared in the presence of these coupling agents or by reaction with an isothiocyanate. In U.S. Pat. No. 4,938,948, imaging of breast tumors, for example, is achieved using monoclonal antibodies, and the detectable imaging moieties are bound to the antibody using linkers such as methyl-p-hydroxybenzimidate or N-succinimidyl-3-(4-hydroxyphenyl)-propionate.

In other embodiments, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature (O'Shannessy et al., 1987).

In other embodiments, immunoglobulins are radiolabeled with nuclides such as tritium. In additional embodiments, nanogold particles (such as sizes from about 0.5 nm-40 nm) and/or Quantum Dots (Hayward, Calif.) are employed.

### IV. Enzymes and Substrates (Chromagens)

Substrates and indicators can be used for detection of ADAMS, such as the exemplary embodiments provided below.

Horseradish peroxidase (HRP) is an enzyme that first forms a complex with hydrogen peroxide and then causes it to decompose, resulting in water and atomic oxygen. Like many other enzymes, HRP and some HRP-like activities can be inhibited by excess substrate. The complex formed between HRP and excess hydrogen peroxide is catalytically inactive and in the absence of an electron donor (e.g. chromogenic substance) is reversibly inhibited. It is the excess hydrogen peroxide and the absence of an electron donor that brings about quenching of endogenous HRP activities.

When used in assays systems, HRP can also be used to convert a defined substrate into its activated chromagen, thus causing a color change. The HRP enzyme may be conjugated to an antibody, protein, peptide, polymer, or other molecule by a number of methods. Such methods are known in the art. Adding glutaraldehyde to a solution containing an admixture of HRP and antibody will result in more antibody molecules being conjugated to each other than to the enzyme. In the two-step procedure, HRP reacts with the bifunctional reagents first. In the second stage, only activated HRP is admixed with the antibody, resulting in much more efficient labelling and no polymerization. HRP is also conjugated to (strept)avidin using the two-step glutaraldehyde procedure. This form is used in procedures where LAB and LSAB are substrate, for example. Conjugation with biotin also involves two steps, as biotin must first be derivatized to the biotinyl-N-hydroxysuccinimide ester or to biotin hydrazide before it can be reacted with the epsilonamino groups of the HRP enzyme.

3,3′-Diaminobenzidine (DAB) is a substrate for enzymes such as HRP that produces a brown end product that is highly insoluble in alcohol and other organic solvents. Oxidation of DAB also causes polymerization, resulting in the ability to react with osmium tetroxide, and thus increasing its staining intensity and electron density. Of the several metals and methods used to intensify the optical density of polymerized DAB, gold chloride in combination with silver sulfide appears to be the most successful.

3-Amino-9-ethylcarbazole (AEC) is a substrate for enzymes such as HRP. Upon oxidation, forms a rose-red end product that is alcohol soluble. Therefore, specimens processed with AEC must not be immersed in alcohol or alcoholic solutions (e.g., Harris' hematoxylin). Instead, an aqueous counterstain and mounting medium should be used. AEC is unfortunately susceptible to further oxidation and, when exposed to excessive light, will fade in intensity. Storage in the dark is therefore recommended.

4-Chloro-1-naphthol (CN) is a substrate for enzymes such as HRP and precipitates as a blue end product. Because CN is soluble in alcohol and other organic solvents, the specimen must not be dehydrated, exposed to alcoholic counterstains, or coverslipped with mounting media containing organic solvents. Unlike DAB, CN tends to diffuse from the site of precipitation.

p -Phenylenediamine dihydrochloride/pyrocatechol (Hanker-Yates reagent) is a an electron donor substrate for enzymes such as HRP and gives a blue-black reaction product that is insoluble in alcohol and other organic solvents. Like polymerized DAB, this reaction product can be osmicated. Varying results have been achieved with Hanker-Yates reagent in immunoperoxidase techniques.

Calf intestine alkaline phosphatase (AP) (molecular weight 100 kD) is an enzyme that removes (by hydrolysis) and transfers phosphate groups from organic esters by breaking the P-0 bond; an intermediate enzyme-substrate bond is briefly formed. The chief metal activators for AP are Mg²⁺, Mn²⁺ and Ca²⁺.

AP had not been used extensively in immunohistochemistry until publication of the unlabeled alkaline phosphataseantialkaline phosphatase (APAAP) procedure. The soluble immune complexes utilized in this procedure have molecular weights of approximately 560 kD. The major advantage of the APAAP procedure compared to the PAP technique is the lack of interference posed by endogenous peroxidase activity. Because of the potential distraction of endogenous peroxidase activity on PAP staining, the APAAP technique is recommended for use on blood and bone marrow smears. Endogenous alkaline phosphatase activity from bone, kidney, liver and some white cells can be inhibited by the addition of 1 mM levamisole to the substrate solution, although 5 mM has been found to be more effective. Intestinal alkaline phosphatases are not adequately inhibited by levamisole.

In the immunoalkaline phosphatase staining method, the enzyme hydrolyzes naphthol phosphate esters (substrate) to phenolic compounds and phosphates. The phenols couple to colorless diazonium salts (chromogen) to produce insoluble, colored azo dyes. Several different combinations of substrates and chromogens have been used successfully.

Naphthol AS-MX phosphate can be used in its acid form or as the sodium salt. The chromogens Fast Red TR and Fast Blue BB produce a bright red or blue end product, respectively. Both are soluble in alcoholic and other organic solvents, so aqueous mounting media must be used. Fast Red TR is preferred when staining cell smears.

Additional exemplary substrates include naphthol AS-BI phosphate, naphthol AS-TR phosphate and 5-bromo-4-chloro-3-indoxyl phosphate (BCIP). Other possible chromogens include Fast Red LB, Fast Garnet GBC, Nitro Blue Tetrazolium (NBT) iodonitrotetrazolium Violet (INT), and derivatives of the structures, for example.

### V. Immunodetection Methods

In still further embodiments, antibodies may be employed to detect wild-type and/or mutant ligand proteins, polypeptides and/or peptides. Wild-type and/or mutant ligand specific antibodies can be used in the methods of the present invention. Some immunodetection methods include flow cytometry, enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, Methods Mol Biol. 1999; 109: 215-37; Gulbis B and Galand P, Hum Pathol. 1993 December; 24(12): 1271-85; and De Jager R et al., Semin Nucl Med. 1993 April; 23(2): 165-79, each incorporated herein by reference.

In general, the immunobinding methods include obtaining a sample suspected of comprising ligand protein, polypeptide and/or peptide, and contacting the sample with a first ligand binding peptide (e.g., an anti-ligand antibody) in accordance with the present invention, as the case may be, under conditions effective to allow the formation of immunocomplexes.

In terms of antigen detection, the biological sample analyzed may be any sample that is suspected of comprising a wild-type or mutant ligand protein-specific antigen, such as a tissue section or specimen, a homogenized tissue extract, biopsy aspirates, a cell, separated and/or purified forms of any of the above wild-type or mutant ADAMS-containing compositions, or even any biological fluid that comes into contact with the tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ligand protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The anti-ligand antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding agent that has binding affinity for the antibody. In these cases, the second binding agent may be linked to a detectable label. The second binding agent is itself often an antibody, which may thus be termed a “secondary” antibody, or a polymer detection system. The primary immune complexes are contacted with the labeled, secondary binding agent, or antibody/polymer detection system, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding agent, such as an antibody, that has binding affinity for the antibody is used to form secondary immune complexes, as described above. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

In another embodiment, a biotinylated monoclonal or polyclonal antibody is used to detect the target antigen(s), and a second step antibody is then used to detect the biotin attached to the complexed biotin. In that method the sample to be tested is first incubated in a solution comprising the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a protein binding (e.g., antibody) conjugate can be produced that is macroscopically visible.

Another known method of immunodetection takes advantage of the immuno-PCR (Polymerase Chain Reaction) methodology. The PCR method uses a DNA/biotin/streptavidin/antibody complex that is washed out with a low pH or high salt buffer that releases the antibody. The resulting wash solution is then used to carry out a PCR reaction with suitable primers with appropriate controls. In specific embodiments, the enormous amplification capability and specificity of PCR can be utilized to detect a single antigen molecule. Such detection may take place in real-time. For example, the use of quantitative real-time PCR is contemplated.

In the clinical diagnosis and/or monitoring of patients with various forms of disease, the detection of a ADAM9 mutant, and/or an alteration in the levels of ADAM9, in comparison to the levels in a corresponding biological sample from a normal subject is indicative of a patient with the disease. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off above which increased detection will be scored as significant and/or positive.

In one embodiment, immunological detection (by immunohistochemistry) of ADAM9 is scored for both intensity and uniformity (percent of stained cells—membrane only, cytoplasmic only or a combination of membrane and cytoplasmic). Comparative scales for ADAM9 expression for intensity correlate as 0—Negative, 0-1—Very Weak, 1—Weak, 1-2—Weak to Moderate, 2—Moderate, 2-3—Moderate to Strong, 3—Strong. Quantitatively, Score 0 represents that no staining (e.g., membrane only, cytoplasmic only or a combination of membrane and cytoplasmic) is observed in tumor cells. A Score 1 represents a faint/barely perceptible staining (e.g., membrane only, cytoplasmic only or a combination of membrane and cytoplasmic) in tumor cells. For Score 2, a moderate staining (e.g., membrane only, cytoplasmic only or a combination of membrane and cytoplasmic) is observed in tumor cells. Lastly, Score 3 represents strong staining (e.g., membrane only, cytoplasmic only or a combination of membrane and cytoplasmic) in the tumor cells.

### VI. ADAM9-Binding Agents

Any antibodies that bind ADAM9 can be used in the detection methods of the present invention. Examples of therapeutically effective anti-ADAM9 antibodies can be found in WO2018/119196 and WO2020/005945, which are herein incorporated by reference. Antibodies that bind to ADAM9 are also commercially available from Abcam, Thermofisher, Sigma-Aldrich, and other companies. The full-length amino acid (aa) and nucleotide (nt) sequences for ADAM9 are known in the art and also provided herein. A specifically useful antibody for detection of ADAM9 is the rabbit monoclonal antibody (Cell Signaling Technologies #41515).

### VII. Anti-ADAM9 Immunoconjugates

The present invention also includes methods for increasing the efficacy of conjugates (also referred to herein as immunoconjugates) comprising the anti-ADAM9 antibodies, antibody fragments, functional equivalents, improved antibodies and their aspects as disclosed herein, linked or conjugated to a cytotoxin (drug) or prodrug. Exemplary ADAM9 immunoconjugates can be found in WO2018/119196 and WO2020/005945, which are herein incorporated by reference.

Suitable drugs or prodrugs are known in the art. In certain embodiments, drugs or prodrugs are cytotoxic agents. The cytotoxic agent used in the cytotoxic conjugate of the present invention can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivaties, leptomycin derivaties, methotrexate, cisplatin, carboplatin, daunorubicin, doxorubicin, vincristine, vinblastine, melphalan, mitomycin C, chlorambucil and morpholino doxorubicin. In certain embodiments, the cytotoxic agents are maytansinoids and maytansinoids analogs.

In some embodiments, the ADAM9 immunoconjutate that can be used in the methods of present disclosure is an immunoconjugate represented by the following formula:

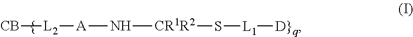

or a pharmaceutically acceptable salt thereof, wherein:

CB is an anti-ADAMS antibody or ADAMS-binding fragment thereof;

L₂ is represented by one of the following formula:

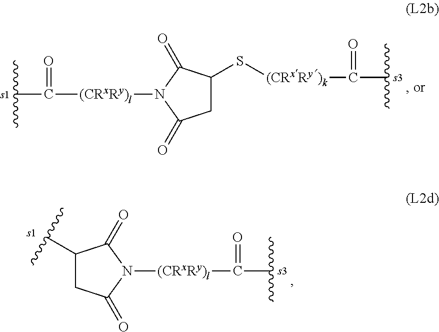

wherein:
R^{x}, R^{y}, R^{x′} and R^{y′}, for each occurrence, are independently H, —OH, halogen, —O-(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;

L₁ is represented by the following formula:

—CR³R⁴—(CH₂)₁₋₈—C(═O)—

wherein R³ and R⁴ are each independently H or Me, and the —C(═O)— moiety in L₁ is connected to D;

D is represented by the following formula:

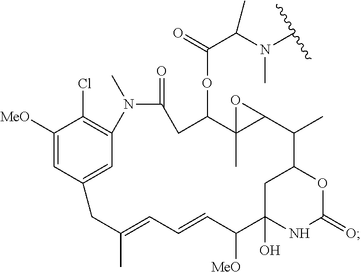 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

q is an integer from 1 to 20.

In some embodiments, for immunoconjugates of formula (I) described above, Rx, Ry, Rx′ and Ry′ are all H; and l and k are each independently an integer an integer from 2 to 6; and the remaining variables are as described above for formula (I).

In some embodiments, for immunoconjugates of formula (I) described above, A is a peptide containing 2 to 5 amino acid residues; and the remaining variables are as described above for formula (I) or in the 1st specific embodiment. In some embodiments, A is a peptide cleavable by a protease. In some embodiments, a peptide cleavable by a protease expressed in tumor tissue. In some embodiments, A is a peptide having an amino acid that is covalent linked with —NH—CR₁R²—S-L₁-D selected from the group consisting of Ala, Arg, Asn, Asp, Cit, Cys, selino-Cys, Gln, Glu, Gly, Ile, Leu, Lys, Met, Phe, Pro, Ser, Thr, Trp, Tyr and Val, each independently as L or D isomer. In some embodiments, the amino acid connected to —NH—CR1R2—S-L1-D is an L amino acid. In some embodiments, A is selected from the group consisting of Gly-Gly-Gly, Ala-Val, Val-Ala, D-Val-Ala, Val-Cit, D-Val-Cit, Val-Lys, Phe-Lys, Lys-Lys, Ala-Lys, Phe-Cit, Leu-Cit, Ile-Cit, Phe-Ala, Phe-N9-tosyl-Arg, Phe-N9-nitro-Arg, Phe-Phe-Lys, D-Phe-Phe-Lys, Gly-Phe-Lys, Leu-Ala-Leu, Ile-Ala-Leu, Val-Ala-Val, Ala-Ala-Ala, D-Ala-Ala-Ala, Ala-D-Ala-Ala, Ala-Ala-D-Ala, Ala-Leu-Ala-Leu (SEQ ID NO:51), (3-Ala-Leu-Ala-Leu (SEQ ID NO: 52), Gly-Phe-Leu-Gly (SEQ ID NO: 53), Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Gln-Val, Asn-Ala, Gln-Phe, Gln-Ala, D-Ala-Pro, and D-Ala-tBu-Gly, wherein the first amino acid in each peptide is connected to L2 group and the last amino acid in each peptide is connected to —NH—CR¹R²—S-L₁-D; and the remaining variables are as described above for formula (I).

In some embodiments, for immunoconjugates of formula (I) described above, R¹ and R² are both H; and the remaining variables are as described above for formula (I).

In some embodiments, for immunoconjugates of formula (I) described above, L₁ is —(CH₂)₄₋₆'C(═O)—; and the remaining variables are as described above for formula (I).

In some embodiments, for immunoconjugates of formula (I) described above, D is represented by the following formula:

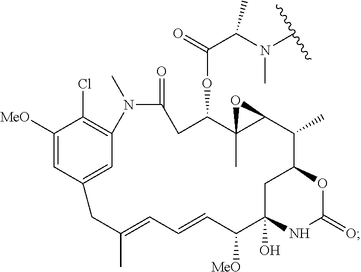 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

and the remaining variables are as described for formula (I).

In certain embodiments, for the immunoconjugate of formula (I), the anti-ADAM9 antibody or ADAMS-binding fragment thereof is a humanized anti-ADAMS antibody or ADAMS-binding fragment thereof.

In certain embodiments, the humanized anti-ADAMS antibody or ADAM9-binding fragment thereof comprises a CDR_{H}¹ domain, a CDR_{H}² domain, a CDR_{H}³ domain a CDR_{L}¹ domain, a CDR_{L}² domain, and a CDR_{L}³ selected from those listed in Tables 1 and 2.

TABLE 1
Exemplary Heavy Chain Variable Region CDR Amino Squences

| CDR_{H}1 | CDR_{H}2 | CDR_{H}3 |
|---|---|---|
| SYWMH (SEQ ID NO: 1) | EIIPIFGHTNYNEKFKS (SEQ ID | GGYYYYGSRDYFDY (SEQ ID |
| (same as SEQ ID NO: 8 in | NO: 3) | NO: 5) |
| WO2020/005945) | (same as SEQ ID NO: 35 in | (same as SEQ ID NO: 10 in |
| | WO2020/005945) | WO2020/005945) |
| SYWIH (SEQ ID NO: 2) | EIIPIFGHTNYNERFQG (SEQ ID | GGYYYYFNSGTLDY (SEQ ID |
| (same as SEQ ID NO: 34 in | NO: 4) | NO: 6) |
| WO2020/005945) | (same as SEQ ID NO: 36 in | (same as SEQ ID NO: 37 in |
| | WO2020/005945) | WO2020/005945) |
| | | GGYYYYIGKGVLDY (SEQ ID |
| | | NO: 7) |
| | | (same as SEQ ID NO: 38 in |
| | | WO2020/005945) |
| | | GGYYYYPRFGWLDY (SEQ ID |
| | | NO: 8) |
| | | (same as SEQ ID NO: 39 in |
| | | WO2020/005945) |
| | | GGYYYYTGKGVLDY (SEQ ID |
| | | NO: 9) |
| | | (same as SEQ ID NO: 40 in |
| | | WO2020/005945) |
| | | GGYYYYDSNAVLDY (SEQ ID |
| | | NO: 10) |
| | | (same as SEQ ID NO: 41 in |
| | | WO2020/005945) |
| | | GGYYYYFHSGTLDY (SEQ ID |
| | | NO: 11) |
| | | (same as SEQ ID NO: 42 in |
| | | WO2020/005945) |
| | | GGYYYYFNKAVLDY (SEQ ID |
| | | NO: 12) |
| | | (same as SEQ ID NO: 43 in |
| | | WO2020/005945) |
| | | GGYYYYGGSGVLDY (SEQ ID |
| | | NO: 13) |
| | | (same as SEQ ID NO: 44 in |
| | | WO2020/005945) |
| | | GGYYYYPRQGFLDY (SEQ ID |
| | | NO: 14) |
| | | (same as SEQ ID NO: 45 in |
| | | WO2020/005945) |
| | | GGYYYYYNSGTLDY (SEQ ID |
| | | NO: 15) |
| | | (same as SEQ ID NO: 46 in |
| | | WO2020/005945) |

TABLE 2
Exemplary Light Chain Variable Region CDR Amino Squences

| CDR_{L}1 | CDR_{L}2 | CDR_{L}3 |
|---|---|---|
| KASQSVDYSGDSYMN (SEQ | AASDLES (SEQ ID NO: 19) | QQSHEDPFT (SEQ ID NO: 20) |
| ID NO: 16) | (same as SEQ ID NO: 13 in | (same as SEQ ID NO: 14 in |
| (same as SEQ ID NO: 62 in | WO2020/005945) | WO2020/005945) |
| WO2020/005945) | | |
| RASQSVDYSGDSYMN (SEQ | | QQSYSTPFT (SEQ ID NO: 21) |
| ID NO: 17) | | (same as SEQ ID NO: 65 in |
| (same as SEQ ID NO: 63 in | | WO2020/005945) |
| WO2020/005945) | | |
| RASQSVDYSGDSYLN (SEQ ID | | |
| NO: 18) | | |
| (same as SEQ ID NO: 64 in | | |
| WO2020/005945) | | |

In certain embodiments, the humanized anti-ADAMS antibody or ADAM9-binding fragment thereof comprises a CDR_{H}1 domain, a CDR_{H}2 domain, and a CDR_{H}3 domain and a CDR_{L}1 domain, a CDR_{L}2 domain, and a CDR_{L}3 domain having the sequences selected from the group consisting of:
(a) SEQ ID NOs: 1, 3, and 5 and SEQ ID NOs: 16, 19, 20, respectively;
(b) SEQ ID NOs: 1, 3, and 5 and SEQ ID NOs: 17, 19, 20, respectively;
(c) SEQ ID NOs: 1, 4, and 5 and SEQ ID NOs: 17, 19, 20, respectively; and
(d) SEQ ID NOs: 2, 4, and 5 and SEQ ID NO: 18, 19, 21, respectively.

In certain embodiments, the anti-ADAMS antibody or ADAMS-binding fragment thereof comprises a CDR_{H}1 domain, a CDR_{H}2 domain, and a CDR_{H}3 domain and a CDR_{L}1 domain, a CDR_{L}2 domain, and a CDR_{L}3 domain having the sequences selected from the group consisting of:
(a) SEQ ID NOs: 1, 3, and 6 and SEQ ID NOs: 16, 19, 20, respectively;
(b) SEQ ID NOs: 1, 3, and 7 and SEQ ID NOs: 16, 19, 20, respectively;
(c) SEQ ID NOs: 1, 3, and 8 and SEQ ID NOs: 16, 19, 20, respectively;
(d) SEQ ID NOs: 1, 3, and 9 and SEQ ID NOs: 16, 19, 20, respectively;
(e) SEQ ID NOs: 1, 3, and 10 and SEQ ID NOs: 16, 19, 20, respectively;
(f) SEQ ID NOs: 1, 3, and 11 and SEQ ID NOs: 16, 19, 20, respectively;
(g) SEQ ID NOs: 1, 3, and 12 and SEQ ID NOs: 16, 19, 20, respectively;
(h) SEQ ID NOs: 1, 3, and 13 and SEQ ID NOs: 16, 19, 20, respectively;
(i) SEQ ID NOs: 1, 3, and 14 and SEQ ID NOs: 16, 19, 20, respectively; and
(j) SEQ ID NOs: 1, 3, and 15 and SEQ ID NOs: 16, 19, 20, respectively.

In certain embodiments, the humanized anti-ADAMS antibody or ADAM9-binding fragment thereof comprises a CDR_{H}1 domain having the sequence of SEQ ID NO: 1, a CDR_{H}2 domain having the sequence of SEQ ID NO: 3, and a CDR_{H}3 domain having the sequence of SEQ ID NO: 5, and a CDR_{L}1 domain having the sequence of SEQ ID NO: 16, a CDR_{L}2 domain having the sequence of SEQ ID NO: 19, and a CDR_{L}3 domain having the sequence of SEQ ID NO: 20.

In certain embodiments, the humanized anti-ADAMS antibody or ADAM9-binding fragment thereof comprises a CDR_{H}1 domain having the sequence of SEQ ID NO: 1, a CDR_{H}2 domain having the sequence of SEQ ID NO: 3, and a CDR_{H}3 domain having the sequence of SEQ ID NO: 14, and a CDR_{L}1 domain having the sequence of SEQ ID NO: 16, a CDR_{L}2 domain having the sequence of SEQ ID NO: 19, and a CDR_{L}3 domain having the sequence of SEQ ID NO: 20.

In some embodiments, the humanized anti-ADAMS antibody or ADAM9-binding fragment thereof comprises a heavy chain variable domain (VH) and a light chain variable domain (VL) having sequences that are at least 90%, at least 95%, or at least 99% identical to sequences selected from the group consisting of:
(a) SEQ ID NO:22 and SEQ ID NO:35, respectively;
(b) SEQ ID NO:22 and SEQ ID NO:36, respectively;
(c) SEQ ID NO:23 and SEQ ID NO:36, respectively; and
(d) SEQ ID NO:24 and SEQ ID NO:37, respectively.

In some embodiments, the humanized anti-ADAMS antibody or ADAM9-binding fragment thereof comprises a heavy chain variable domain (VH) and a light chain variable domain (VL) having sequences selected from the group consisting of:
(a) SEQ ID NO:22 and SEQ ID NO:35, respectively;
(b) SEQ ID NO:22 and SEQ ID NO:36, respectively;
(c) SEQ ID NO:23 and SEQ ID NO:36, respectively; and
(d) SEQ ID NO:24 and SEQ ID NO:37, respectively.

In certain embodiments, the humanized anti-ADAMS antibody or ADAMS-binding fragment thereof comprises a heavy chain variable domain (VH) and a light chain variable domain (VL) having sequences that are at least 90%, at least 95%, or at least 99% identical to sequences selected from the group consisting of:
(a) SEQ ID NO:25 and SEQ ID NO:35, respectively;
(b) SEQ ID NO:26 and SEQ ID NO:35, respectively;
(c) SEQ ID NO:27 and SEQ ID NO:35, respectively;
(d) SEQ ID NO:28 and SEQ ID NO:35, respectively;
(e) SEQ ID NO:29 and SEQ ID NO:35, respectively;
(f) SEQ ID NO:30 and SEQ ID NO:35, respectively;
(g) SEQ ID NO:31 and SEQ ID NO:35, respectively;
(h) SEQ ID NO:32 and SEQ ID NO:35, respectively;
(i) SEQ ID NO:33 and SEQ ID NO:35, respectively; and
(j) SEQ ID NO:34 and SEQ ID NO:35, respectively.

In certain embodiments, the humanized anti-ADAMS antibody or ADAMS-binding fragment thereof comprises a heavy chain variable domain (VH) and a light chain variable domain (VL) having the sequences selected from the group consisting of:
(a) SEQ ID NO:25 and SEQ ID NO:35, respectively;
(b) SEQ ID NO:26 and SEQ ID NO:35, respectively;
(c) SEQ ID NO:27 and SEQ ID NO:35, respectively;
(d) SEQ ID NO:28 and SEQ ID NO:35, respectively;
(e) SEQ ID NO:29 and SEQ ID NO:35, respectively;
(f) SEQ ID NO:30 and SEQ ID NO:35, respectively;
(g) SEQ ID NO:31 and SEQ ID NO:35, respectively;
(h) SEQ ID NO:32 and SEQ ID NO:35, respectively;
(i) SEQ ID NO:33 and SEQ ID NO:35, respectively; and
(j) SEQ ID NO:34 and SEQ ID NO:35, respectively.

In certain embodiments, the humanized anti-ADAMS antibody or ADAMS-binding fragment thereof comprises a heavy chain variable domain (VH) having the sequence of SEQ ID NO: 22 and a light chain variable domain (VL) having the sequence of SEQ ID NO: 35.

In certain embodiments, the humanized anti-ADAMS antibody or ADAMS-binding fragment thereof comprises a heavy chain variable domain (VH) having the sequence of SEQ ID NO: 33 and a light chain variable domain (VL) having the sequence of SEQ ID NO: 35.

TABLE 3
Exemplary Heavy Chain Variable Region and Light Chain Variable Region
Amino Acid Sequences

| | |
|---|---|
| VH | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | YYYYGSRDYF DYWGQGTTVT VSS (SEQ ID NO: 22) |
| | (same as SEQ ID NO: 17 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | YYYYGSRDYF DYWGQGTTVT VSS (SEQ ID NO: 23) |
| | (same as SEQ ID NO: 18 in WO2020/005945) |
| | EVQLVESGGG LVKPGGSLRL SCAASGFTFS [symbol] WVRQA PGKGLEWVGE |
| | [symbol] RFTI SLDNSKNTLY LQMGSLRAED TAVYYCARGG |
| | YYYYGSRDYF DYWGQGTTVT VSS (SEQ ID NO: 24) |
| | (same as SEQ ID NO: 19 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 25) |
| | (same as SEQ ID NO: 20 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 26) |
| | (same as SEQ ID NO: 21 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 27) |
| | (same as SEQ ID NO: 22 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 28) |
| | (same as SEQ ID NO: 23 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 29) |
| | (same as SEQ ID NO: 24 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 30) |
| | (same as SEQ ID NO: 25 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 31) |
| | (same as SEQ ID NO: 26 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 32) |
| | (same as SEQ ID NO: 27 in WO2020/005945) |
| | EVQLVESGGG LVKPGGSLRL SCAASGFTFS SYWMHWVRQA |
| | PGKGLEWVGE [symbol] RFTI SLDNSKNTLY LQMGSLRAED |
| | TAVYYCAR [symbol] WGQGTTVT VSS (SEQ ID NO: 33) |
| | (same as SEQ ID NO: 28 in WO2020/005945) |
| | EVQLVESGGG LVKPGGSLRL SCAASGFTFS SYWMHWVRQA PGKGLEWVGE |
| | [symbol] RFTI SLDNSKNTLY LQMGSLRAED TAVYYCARGG |
| | [symbol] WGQGTTVT VSS (SEQ ID NO: 34) |
| | (same as SEQ ID NO: 29 in WO2020/005945) |
| VL | DIVMTQSPDS LAVSLGERAT ISC [symbol] WY QQKPGQPPKL |
| | LIYAASDLES GIPARFSGSG SGTDFTLTIS SLEPEDFATY YCQQSHEDPF |
| | TFGQGTKLEI K (SEQ ID NO: 35) |
| | (same as SEQ ID NO: 55 in WO2020/005945) |
| | DIVMTQSPDS LAVSLGERAT ISC [symbol] WY QQKPGQPPKL |
| | LIYAASDLES GIPARFSGSG SGTDFTLTIS SLEPEDFATY YCQQSHEDPF |
| | TFGQGTKLEI K (SEQ ID NO: 36) |
| | (same as SEQ ID NO: 56 in WO2020/005945) |
| | DIVMTQSPDS LAVSLGERAT ISC [symbol] WY QQKPGQPPKL |
| | LIYAASDLES GIPARFSGSG SGTDFTLTIS SLEPEDFATY YC [symbol] |
| | TFGQGTKLEI K (SEQ ID NO: 37) |
| | (same as SEQ ID NO: 57 in WO2020/005945) |

In certain embodiments, the humanized anti-ADAMS antibody is a full length antibody comprising an Fc region. In some embodiments, the Fc region is a variant Fc region that comprises:
(a) one or more amino acid modification(s) that reduces(s) the affinity of the variant Fc region for an FcγR selected from the group consisting of: L234A, L235A, and L234A and L235A; and/or
(b) an amino acid modification that introduces a cysteine residue at S442, wherein said numbering is that of the EU index as in Kabat; and/or
(c) one or more amino acid substitution(s) that extend(s) the half-life of the variant Fc region for FcRn selected from the group consisting of: M252Y, S254T, and T256E

In some embodiments, the humanized anti-ADAMS antibody comprises a heavy chain and a light chain having the sequences selected from the group consisting of:
(a) SEQ ID NO:38 and SEQ ID NO:50, respectively;
(b) SEQ ID NO:39 and SEQ ID NO:50, respectively; and
(c) SEQ ID NO:40 and SEQ ID NO:50, respectively.

In certain embodiments, the humanized anti-ADAMS antibody comprises a heavy chain and a light chain having the sequences selected from the group consisting of:
(a) SEQ ID NO:41 and SEQ ID NO:50, respectively;
(b) SEQ ID NO:42 and SEQ ID NO:50, respectively;
(c) SEQ ID NO:43 and SEQ ID NO:50, respectively;
(d) SEQ ID NO:44 and SEQ ID NO:50, respectively;
(e) SEQ ID NO:45 and SEQ ID NO:50, respectively;
(f) SEQ ID NO:46 and SEQ ID NO:50, respectively; and
(g) SEQ ID NO:47 and SEQ ID NO:50, respectively.

In certain embodiments, the humanized/optimized anti-ADAMS antibody comprises a heavy chain having the sequence of SEQ ID NO: 40 and a light chain having the sequence of SEQ ID NO:50.

In certain embodiments, the humanized/optimized anti-ADAMS antibody comprises a heavy chain having the sequence of SEQ ID NO:45 and a light chain having the sequence of SEQ ID NO:50.

In certain embodiments, X in SEQ ID NO:41, SEQ ID NO:42, SEQ ID NO:43, SEQ ID NO:44, SEQ ID NO:45, SEQ ID NO:46 or SEQ ID NO:47 is lysine.

In certain embodiments, X in SEQ ID NO:41, SEQ ID NO:42, SEQ ID NO:43, SEQ ID NO:44, SEQ ID NO:45, SEQ ID NO:46 or SEQ ID NO:47 is absent.

In certain embodiments, the humanized anti-ADAMS antibody comprises a heavy chain and a light chain having the sequences of SEQ ID NO:49 and SEQ ID NO:50, respectively. In certain embodiments, the humanized anti-ADAMS antibody comprises a heavy chain and a light chain having the sequences of SEQ ID NO:48 and SEQ ID NO:50, respectively.

TABLE 4
Full-Length Heavy Chain and Light Chain Amino Acid Sequences

| | |
|---|---|
| Heavy | EVQLVESGGGLVKPGGSLRLSCAASGFTFSSYWMHWVRQAPGKGLEWVGEIIPIFGHTNYNEKFK |
| Chain | SRFTISLDNSKNTLYLQMGSLRAEDTAVYYCARGGYYYYGSRDYFDYWGQGTTVTVSSASTKGPS |
| | VFPLAPSSKSTSGGTAALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLIVLHQDWLNGKEY |
| | KCKVSNKALPAPIEKTISKAKGQPREPQVYTLPPSREEMTKNQVSLTCLVKGFYPSDIAVEWESN |
| | GQPENNYKTTPPVLDSDGSFFLYSKLTVDKSRWQQGNVFSCSVMHEALHNHYTQKSLSLSPGX |
| | (SEQ ID No: 38) |
| | X is a lysine (K) or is absent |
| | (same as SEQ ID NO: 50 in WO2020/005945) |
| | EVQLVESGGGLVKPGGSLRLSCAASGFTFSSYWMHWVRQAPGKGLEWVGEIIPIFGHTNYNEKFK |
| | SRFTISLDNSKNTLYLQMGSLRAEDTAVYYCARGGYYYYPRFGWLDYWGQGTTVTVSSASTKGPS |
| | VFPLAPSSKSTSGGTAALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLIVLHQDWLNGKEY |
| | KCKVSNKALPAPIEKTISKAKGQPREPQVYTLPPSREEMTKNQVSLTCLVKGFYPSDIAVEWESN |
| | GQPENNYKTTPPVLDSDGSFFLYSKLTVDKSRWQQGNVFSCSVMHEALHNHYTQKSLSLSPGX |
| | (SEQ ID No: 39) |
| | X is a lysine (K) or is absent |
| | (same as SEQ ID NO: 51 in WO2020/005945) |
| | EVQLVESGGGLVKPGGSLRLSCAASGFTFSSYWMHWVRQAPGKGLEWVGEIIPIFGHTNYNEKFK |
| | SRFTISLDNSKNTLYLQMGSLRAEDTAVYYCARGGYYYYPRQGFLDYWGQGTTVTVSSASTKGPS |
| | VFPLAPSSKSTSGGTAALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSREEMTKNQVSLTCLVKGFYPSDIAVEWESN |
| | GQPENNYKTTPPVLDSDGSFFLYSKLTVDKSRWQQGNVFSCSVMHEALHNHYTQKSLSLSPGX |
| | (SEQ ID No: 40) |
| | X is a lysine (K) or is absent |
| | (same as SEQ ID NO: 52 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEA |
| | AGGPSVFLFP PKPKDTLMIS RTPEVTCVVV DVSHEDPEVK FNWYVDGVEV HNAKTKPREE |
| | QYNSTYRVVS VLIVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLSLS PGX (SEQ ID No: 41) |
| | wherein X is a lysine (K) or is absent |
| | (same as SEQ ID NO: 141 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEL |
| | LGGPSVFLFP PKPKDTLMIS RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLCLS PGX (SEQ ID No: 42) |
| | wherein X is a lysine (K) or is absent |
| | (same as SEQ ID NO: 142 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEA |
| | AGGPSVFLFP PKPKDTLMIS RTPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLCLS PGX (SEQ ID No: 43) |
| | wherein X is a lysine (K) or is absent |
| | (same as SEQ ID NO: 143 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEL |
| | LGGPSVFLFP PKPKDTLYIT REPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLSLS PGX (SEQ ID No: 44) |
| | wherein X is a lysine (K) or is absent |
| | (same as SEQ ID NO: 151 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEL |
| | LGGPSVFLFP PKPKDTLYIT REPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLCLS PGX (SEQ ID No: 45) |
| | wherein X is a lysine (K) or is absent |
| | (same as SEQ ID NO: 152 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEA |
| | AGGPSVFLFP PKPKDTLYIT REPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLSLS PGX (SEQ ID No: 46) |
| | wherein X is a lysine (K) or is absent |
| | (same as SEQ ID NO: 153 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGEL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEA |
| | AGGPSVFLFP PKPKDTLYIT REPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLCLS PGX (SEQ ID No: 47) |
| | wherein X is a lysine (K) or is absent |
| | (same as SEQ ID NO: 154 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGEL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEL |
| | LGGPSVFLFP PKPKDTLYIT REPEVTCVVV DVSHEDPEVK FNWYVDGVEV HNAKTKPREE |
| | QYNSTYRVVS VLIVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLSLS PG (SEQ ID NO: 48) |
| | (same as SEQ ID NO: 155 in WO2020/005945) |
| | EVQLVESGGG LVKPGGSLRL SCAASGFTFS SYWMHWVRQA PGKGLEWVGE IIPIFGHTNY |
| | NEKFKSRFTI SLDNSKNTLY LQMGSLRAED TAVYYCARGG YYYYPRQGFL DYWGQGTTVT |
| | VSSASTKGPS VFPLAPSSKS TSGGTAALGC LVKDYFPEPV TVSWNSGALT SGVHTFPAVL |
| | QSSGLYSLSS VVTVPSSSLG TQTYICNVNH KPSNTKVDKR VEPKSCDKTH TCPPCPAPEL |
| | LGGPSVFLFP PKPKDTLYIT REPEVTCVVV DVSHEDPEVK FNWYVDGVEV HNAKTKPREE |
| | QYNSTYRVVS VLTVLHQDWL NGKEYKCKVS NKALPAPIEK TISKAKGQPR EPQVYTLPPS |
| | REEMTKNQVS LTCLVKGFYP SDIAVEWESN GQPENNYKTT PPVLDSDGSF FLYSKLTVDK |
| | SRWQQGNVFS CSVMHEALHN HYTQKSLCLS PG (SEQ ID NO: 49) |
| | (same as SEQ ID NO: 156 in WO2020/005945) |
| Light | DIVMTQSPDS LAVSLGERAT ISCKASQSVD YSGDSYMNWY QQKPGQPPKL LIYAASDLES |
| Chain | GIPARFSGSG SGTDFTLTIS SLEPEDFATY YCQQSHEDPF TFGQGTKLEI KRTVAAPSVE |
| | IFPPSDEQLK SGTASVVCLL NNFYPREAKV QWKVDNALQS GNSQESVTEQ DSKDSTYSLS |
| | STLTLSKADY EKHKVYACEV THQGLSSPVT KSENRGEC (SEQ ID NO: 50) |
| | (same as SEQ ID NO: 68 in WO2020/005945) |

In certain embodiments, the anti-ADAMS immunoconjugate of the present invention is represented by the following formula:

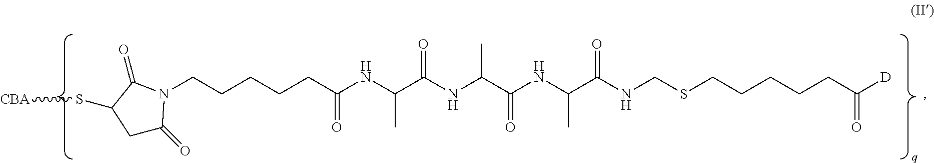

wherein:
   CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
   q is 1 or 2;
   D is represented by the following formula:

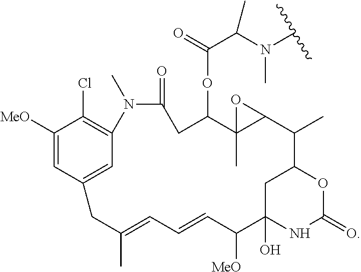 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

In certain embodiments, the anti-ADAMS immunoconjugate of the present invention is represented by the following formula:

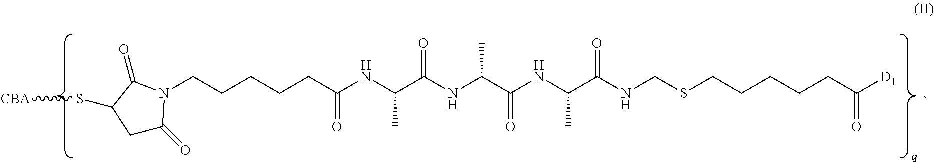

wherein:
   CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
q is 1 or 2;
D₁ is represented by the following formula:

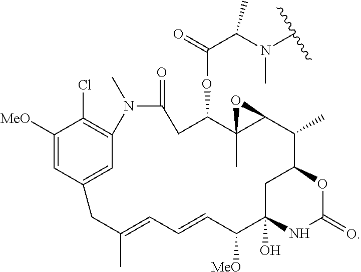 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the humanized anti-ADAMS antibody or ADAM9-binding fragment thereof comprises a heavy chain variable domain (VH) and a light chain variable domain (VL) having sequences of SEQ ID NO:33 and SEQ ID NO:35, respectively. In some embodiments, the humanized anti-ADAMS antibody comprises a heavy chain and a light chain having the sequences of SEQ ID NO:42 and SEQ ID NO:50, respectively. In some embodiments, the humanized anti-ADAMS antibody comprises a heavy chain and a light chain having the sequences of SEQ ID NO:45 and SEQ ID NO:50, respectively. In some embodiments, In some embodiments, X in SEQ ID NO:42 or SEQ ID NO:45 is lysine. In some embodiments, In some embodiments, X in SEQ ID NO:42 or SEQ ID NO:45 is absent. In some embodiments, the humanized anti-ADAMS antibody comprises a heavy chain and a light chain having the sequences of SEQ ID NO:49 and SEQ ID NO:50, respectively. In some embodiments, the DAR value for a composition (e.g., pharmaceutical compositions) comprising the immunoconjugate is in the range of 1.0 to 2.5, 1.5 to 2.5, 1.8 to 2.2, or 1.9 to 2.1. In some embodiments, the DAR is 1.8, 1.9, 2.0 or 2.1.

In certain embodiments, the anti-ADAMS immunoconjugate is represented by the following formula:

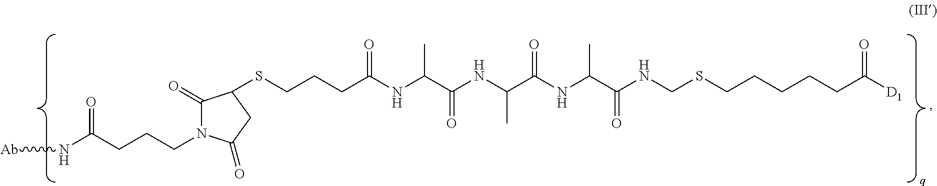

wherein:
   CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
   q is an integer from 1 or 10;
   D is represented by the following formula:

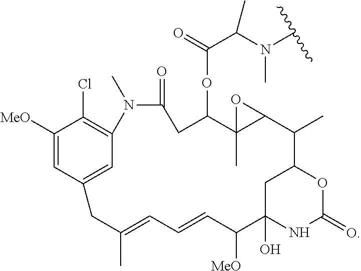 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]

In certain embodiments, the anti-ADAMS immunoconjugate is represented by the following formula:

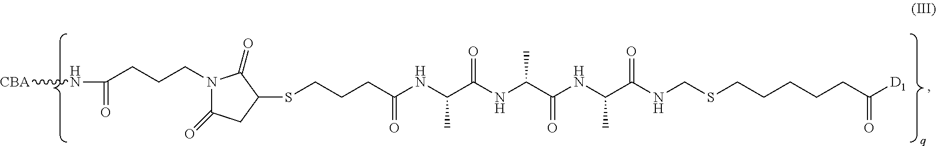

wherein:
   CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
   q is an integer from 1 or 10;
   ₁ is represented by the following formula:

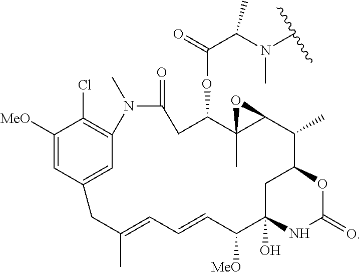 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, the humanized anti-ADAMS antibody or ADAM9-binding fragment thereof comprises a heavy chain variable domain (VH) and a light chain variable domain (VL) having sequences of SEQ ID NO:33 and SEQ ID NO:35, respectively. In some embodiments, the humanized anti-ADAMS antibody comprises a heavy chain and a light chain having the sequences of SEQ ID NO:40 and SEQ ID NO:50, respectively. In some embodiments, the humanized anti-ADAMS antibody comprises a heavy chain and a light chain having the sequences of SEQ ID NO:44 and SEQ ID NO:50, respectively. In some embodiments, X in SEQ ID NO:40 and SEQ ID NO:44 is lysine. In some embodiments, X in SEQ ID NO:40 and SEQ ID NO:44 is absent. In some embodiments, the humanized anti-ADAM9 antibody comprises a heavy chain and a light chain having the sequences of SEQ ID NO:48 and SEQ ID NO:50, respectively. In some embodiments, the DAR value for a composition (e.g., pharmaceutical compositions) comprising the immunoconjugate is in the range of 1.0 to 5.0, 1.0 to 4.0, 1.5 to 4.0, 2.0 to 4.0, 2.5 to 4.0, 2.9 to 3.3, 3.3 to 3.8, 1.5 to 2.5, or 1.8 to 2.2. In some embodiments, the DAR is less than 4.0, less than 3.8, less than 3.6, less than 3.5, less than 3.0 or less than 2.5. In some embodiments, the DAR is in the range of 3.0 to 3.2. In some embodiments, the DAR is in the range of 3.5 to 3.7. In some embodiments, the DAR is 3.1, 3.2, 3.3, 3.4, 3.5, 3.6 or 3.7. In some embodiments, the DAR is in the range of 1.9 to 2.1. In some embodiments, the DAR is 1.9, 2.0 or 2.1.

In certain embodiments, the anti-ADAMS immunoconjugate comprising an humanized anti-ADAMS antibody covalently linked to a maytansinoid compound DM21-C represented by the following formula:

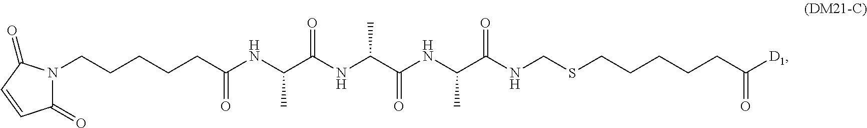

wherein D₁ has the structure of:

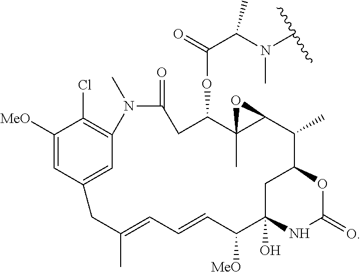 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

The humanized anti-ADAMS antibody comprises a heavy chain and a light chain having the sequences of SEQ ID NO:45 and SEQ ID NO:50, respectively. In certain embodiments, the anti-ADAMS immunoconjugate is IMGC936 represented by the following structure:

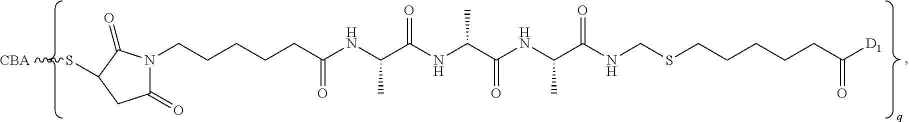

wherein:
   CBA is a humanized anti-ADAMS antibody comprising a heavy chain and a light chain having the sequences of SEQ ID NO:45 and SEQ ID NO:50, respectively; and;
   q is 2;
   D₁ is represented by the following formula:

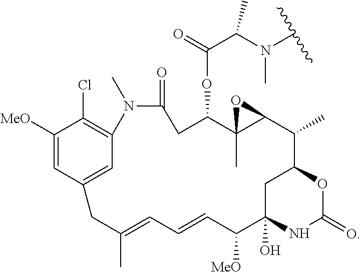 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

### VIII. Pharmaceutical Compositions and Therapeutic Methods

As provided herein, the anti-ADAM9 immunoconjugates of the present invention, comprising the humanized anti-ADAM9 antibodies or ADAM9-binding fragment thereof provided herein, have the ability to bind ADAM9 present on the surface of a cell and mediate cell killing. In particular, the immunoconjugates of the present invention comprising a pharmacological agent, are internalized and mediate cell killing via the activity of the pharmacological agent. Such cell killing activity may be augmented by the immunoconjugate inducing antibody-dependent cell-mediated cytotoxicity (ADCC) and/or complement dependent cytotoxicity (CDC).

Thus, immunoconjugates of the present invention have the ability to treat any disease or condition associated with or characterized by the expression of ADAM9. As discussed above, ADAM9 is an onco-embryonic antigen expressed in numerous blood and solid malignancies that is associated with high-grade tumors exhibiting a less-differentiated morphology, and is correlated with poor clinical outcomes. Thus, without limitation, the immunoconjugates of the present invention may be employed in the treatment of cancer, particularly a cancer characterized by the expression of ADAM9.

In other particular embodiments, immunoconjugates of the present invention may be useful in the treatment of lung cancer (e.g., non-small-cell lung cancer), colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer. In other particular embodiments, immunoconjugates of the present invention may be useful in the treatment of non-small-cell lung cancer, colorectal cancer, gastric cancer, breast cancer (e.g., triple negative breast cancer (TNBC)), or pancreatic cancer.

In further embodiments, immunoconjugates of the present invention may be useful in the treatment of non-small-cell lung cancer (squamous cell, nonsquamous cell, adenocarcinoma, or large-cell undifferentiated carcinoma), colorectal cancer (adenocarcinoma, gastrointestinal carcinoid tumors, gastrointestinal stromal tumors, primary colorectal lymphoma, leiomyosarcoma, or squamous cell carcinoma) or breast cancer (e.g., triple negative breast cancer (TNBC)).

In addition to their utility in therapy, the immunoconjugates of the present invention may be detectably labeled and used in the diagnosis of cancer or in the imaging of tumors and tumor cells.

The compositions of the invention include bulk drug compositions useful in the manufacture of pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of the immunoconjugates of the present invention, or a combination of such agents and a pharmaceutically acceptable carrier. Preferably, compositions of the invention comprise a prophylactically or therapeutically effective amount of immunoconjugates of the present invention and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Generally, the ingredients of compositions of the invention are supplied either separately or mixed together in unit dosage form, for example, as a liquid, a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with an immunoconjugates of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. A kit can comprise any of the immunoconjugates of the present invention. The kit can further comprise one or more other prophylactic and/or therapeutic agents useful for the treatment of cancer, in one or more containers.

The compositions of the present invention may be provided for the treatment, prophylaxis, and amelioration of one or more symptoms associated with a disease, disorder by administering to a subject an effective amount an immunoconjugate of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262: 4429-4432), construction of a nucleic acid as part of a retroviral or other vector, etc.

Methods of administering an immunoconjugate of the invention include, but are not limited to, parenteral administration (e.g., intradermal, intramuscular, intraperitoneal, intravenous and subcutaneous), epidural, and mucosal (e.g., intranasal and oral routes). In a specific embodiment, the immunoconjugates of the present invention are administered intramuscularly, intravenously, or subcutaneously. The compositions may be administered by any convenient route, for example, by infusion or bolus injection, and may be administered together with other biologically active agents. Administration can be systemic or local.

The invention also provides that preparations of the immunoconjugates of the present invention are packaged in a hermetically sealed container such as a vial, an ampoule or sachette indicating the quantity of the molecule. In one embodiment, such molecules are supplied as a liquid, a dry sterilized lyophilized powder or water free concentrate in a hermetically sealed container and can be reconstituted, e.g., with water or saline to the appropriate concentration for administration to a subject. Preferably, the immunoconjugates of the present invention are supplied as a dry sterile lyophilized powder in a hermetically sealed container. Preferably, the immunoconjugates of the present invention are supplied as a liquid in a hermetically sealed container.

The lyophilized preparations of the immunoconjugates of the present invention should be stored at between 2° C. and 8° C. in their original container and the molecules should be administered within 12 hours, preferably within 6 hours, within 5 hours, within 3 hours, or within 1 hour after being reconstituted. In an alternative embodiment, such molecules are supplied in liquid form in a hermetically sealed container indicating the quantity and concentration of the molecule, fusion protein, or conjugated molecule. Preferably, such immunoconjugates when provided in liquid form are supplied in a hermetically sealed container.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering an immunoconjugate of the invention, care must be taken to use materials to which the molecule does not absorb.

The compositions of the invention can be delivered in a vesicle, in particular a liposome (See Langer (1990) “New Methods Of Drug Delivery,” Science 249: 1527-1533); Treat et al., in LIPOSOMES IN THE THERAPY OF INFECTIOUS DISEASE AND CANCER, Lopez-Berestein and Fidler (eds.), Liss, New York, pp. 353- 365 (1989); Lopez-Berestein, ibid., pp. 3 17-327).

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### PDX Materials and Methods

Animal Models: Patient derived tumor samples were implanted in 6-8 weeks old athymic Nude-Foxnlnu mice. When tumors reached an average tumor volume of 100-300 mm³ animals were matched by tumor volume into treatment or control groups to be used for dosing. Mice were dosed with either vehicle, non-targeting ADC (0.1 mg/kg of maytansinoid DM payload), or IMGC936 (anti-ADAMS immunoconjugate, 8.6 mg/kg of ADC or 0.1 mg/kg of maytansinoid DM payload) by intravenous injection. Tumor measurements and weights were taken two times per week.

Efficacy: Anti-tumor activity was defined by NCI standards: mean tumor volume of treated mice over control mice (%) >42% (inactive), <42% (active), and <10% (highly active).

### IHC Materials and Methods

Tissue samples: All tissue samples used are FFPE (Formalin fixed & paraffin embedded) whole tissue samples, except when noted for esophageal cancer, some were tissue cores from a tissue microarray (TMA).

### Immunohistochemistry

A research use only immunohistochemical (IHC) assay for ADAM9 was carried out using the Ventana Discovery Ultra autostainer. The primary antibody for ADAM9 was a rabbit monoclonal antibody purchased from Cell Signaling Technologies (Cat #4151S). This assay was used for PDX studies. The IHC protocol is described briefly: Antigen recovery was conducted using 32-minute incubation conditions with CC1 buffer (VMSI, Catalog No. 950-224). One drop of inhibitor CM was added and incubated for 8 minutes. Slides were incubated with a 1:100 dilution (2.5 ug/mL) of the stock concentration of the primary ADAM9 antibody (0.25 mg/mL) in diluent (VMSI, 760-219) for 32 minutes at 37° C. As negative control, specimens were incubated with an IgG rabbit antibody (Vector Cat #I-1000) under the same conditions. Enzymatic detection of anti-ADAM9 antibody was accomplished with anti-rabbit IgG conjugated to HQ (VMSI, Catalog No. 760-4815), followed by an anti-HQ conjugated to HRP (VMSI, Catalog No. 760-2020) which were incubated for 12 minutes. Anti-ADAM9 antibody was detected using the ChromoMap DAB detection kit (VMSI, Catalog No. 760-159). Chromogen is deposited by a reaction with hydrogen peroxide in the presence of diaminobenzidine (DAB) and copper sulfate.

An immunohistochemical (IHC) assay for the detection of ADAM9 in FFPE samples was developed and validated at Roche Tissue Diagnostics (RTD). The Robust Prototype Assay (RPA) uses a Spring Bioscience anti-ADAM9 (J12H2L3) antibody with Ventana OptiView DAB detection on the Ventana Benchmark ULTRA staining platform. High quality staining was observed in a membranous and cytoplasmic pattern in accordance with published findings. The RPA assay demonstrated consistent staining across a variety of assay parameters throughout the development process and met standards of specificity, accuracy, and analytical precision.

The IHC protocol is described briefly: Antigen recovery was conducted using 92-minute incubation conditions with CC1 buffer (VMSI, Catalog No. 950-224). Slides were incubated with a 1:282 dilution of the stock concentration of the primary antibody in diluent 90103 (VMSI) for 16 minutes at 36° C. Stock antibody concentration refers to the concentration at which the antibody was provided to Ventana by the manufacturer. As negative control, specimens were incubated with an IgG rabbit monoclonal antibody under the same conditions. Anti- ADAM9 antibody was detected using the OptiView™ detection kit (VMSI, Catalog No. 760-700). Enzymatic detection of anti-ADAM9 antibody was accomplished with secondary goat anti-mouse and anti-rabbit IgG conjugated to HQ, followed by an anti-HQ conjugated to HRP. Chromogen is deposited by a reaction with hydrogen peroxide in the presence of diaminobenzidine (DAB) and copper sulfate. The secondary antibody, HRP multimer, and all chromogen reagents were applied at the instrument's default times.

Scoring and data analysis: Staining intensity was scored on a semi-quantitative integer scale from 0 (negative) to 3 by a certified anatomic pathologist. The percentage of cells staining positively for cytoplasm, membrane, and cytoplasm and/or membrane (cyto-membrane) at each intensity level was recorded. Scoring was based on localization of ADAM9 to the cell membrane and cytoplasm. In tumor samples, viable malignant cells were scored.

H-scores were calculated. The H-score combines components of staining intensity with the percentage of positive cells. It has a value between 0 and 300 and is defined as:

1*(percentage of cells staining at intensity category 1)+2*(percentage of cells staining at intensity category 2)+3*(percentage of cells staining at intensity category 3)=H score

### Example 1. PDX Study

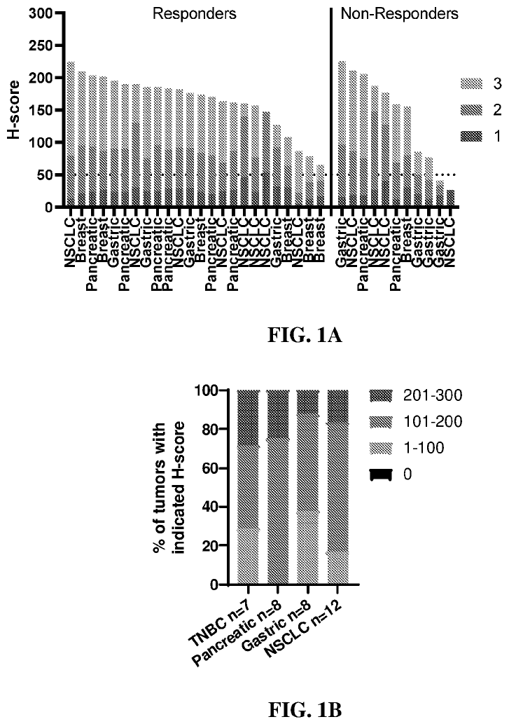

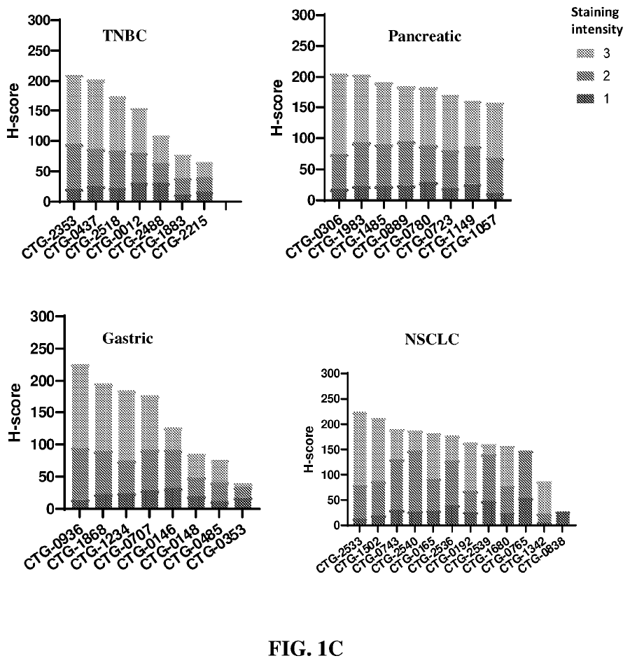

Activity of IMGC936 was analyzed in PDX models derived from adenocarcinoma non-small cell lung carcinoma (NSCLC), triple negative breast cancer (TNBC), pancreatic cancer, and gastric cancer tumors. The range of ADAM9 expression was 27 to 226 by H-score, with 80% of the samples having H-scores above 101 (Table 5). A single dose of IMGC936 at 0.1 mg/kg (8.6 mg/kg by antibody) was well tolerated. Across the tumor types tested, IMGC936 was active or highly active in 24 out of 35 models (69%) with complete regressions in 6 models (4 NSCLC, 2 TNBC). The 24 IMGC936 sensitive models had H-scores between 65 and 224. The 11 non-sensitive models had H-scores between 27 and 226. All PDX models in this study with H-score above 50 responded to IMGC936 (FIG. 1A). ADAM9 prevalance data for each cancer type is shown in FIG. 1B. H-scores and staining intensities for each PDX model are shown in FIG. 1C.

TABLE 5
Results from NSCLC- Adenocarcinoma, Gastric
Cancer, TNBC, and Pancreatic PDX Models

| | | Study Tissue | | |
|---|---|---|---|---|
| Indication | Model | H-Score | Outcome | Response Rate |
| NSCLC- | CTG-2533 | 224 | Highly active | 8/12 |
| Adeno | CTG-1502 | 211 | Inactive | |
| | CTG-0165 | 182 | Highly active | |
| | CTG-0192 | 163 | Active | |
| | CTG-1680 | 157 | Active | |
| | CTG-2539 | 160 | Highly active | |
| | CTG-0765 | 147 | Highly active | |
| | CTG-0743 | 190 | Highly active | |
| | CTG-0838 | 27 | Inactive | |
| | CTG-2536 | 177 | Inactive | |
| | CTG2540 | 187 | Inactive | |
| | CTG-1342 | 87 | Active | |
| Gastric | CTG-0148 | 85 | Inactive | 4/8 |
| Cancer | CTG-0146 | 127 | Active | |
| | CTG-1868 | 195 | Active | |
| | CTG-0485 | 77 | Inactive | |
| | CTG-0707 | 176 | Active | |
| | CTG-0936 | 226 | Inactive | |
| | CTG-1234 | 185 | Active | |
| | CTG-0353 | 41 | Inactive | |
| TNBC | CTG-2353 | 209 | Active | 6/7 |
| | CTG-1883 | 78 | Highly active | |
| | CTG-0437 | 202 | Highly active | |
| | CTG-2215 | 65 | Active | |
| | CTG-2518 | 173 | Active | |
| | CTG-2488 | 108 | Highly active | |
| | CTG-0012 | 155 | Inactive | |
| Pancreatic | CTG-0780 | 183 | Active | 6/8 |
| Cancer | CTG-1485 | 190 | Active | |
| | CTG-1983 | 203 | Active | |
| | CTG-0889 | 185 | Active | |
| | CTG-0723 | 170 | Active | |
| | CTG-1057 | 158 | Inactive | |
| | CTG-1149 | 162 | Active | |
| | CTG-0306 | 205 | Inactive | |

### Example 2. ADAM9 Prevalence

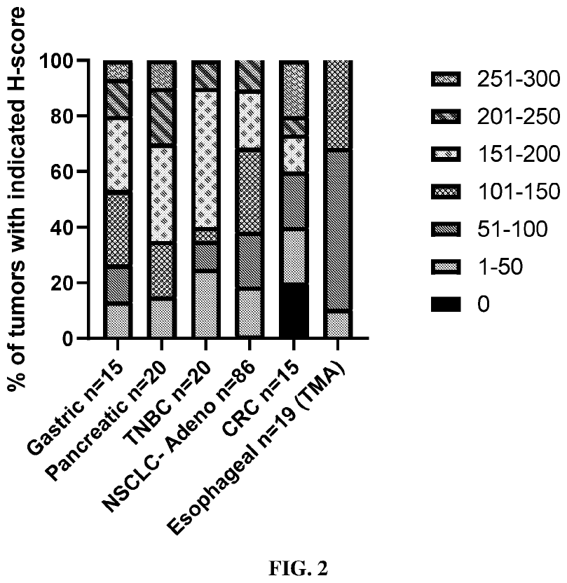

ADAM9 was highly expressed in multiple tumor types. A majority of the tumor samples had medium to high levels of ADAM9 with 62% of adenocarcinoma NSCLC, 65% of TNBC, 73% of gastric cancer, and 85% of pancreatic cancer samples having H- scores of 101 to 300 (FIG. 2). ADAM9 expression in colorectal cancer (CRC) and esophageal cancer was also investigated. ADAM9 expression was lower in CRC and esophageal cancers with 40% and 32%, respectively of samples having an H-score above 100. The remaining tumor samples had lower levels of ADAM9 expression (H-score 1 to 100) with 1.2% of NSCLC and 20% of CRC samples being ADAM9-negative.

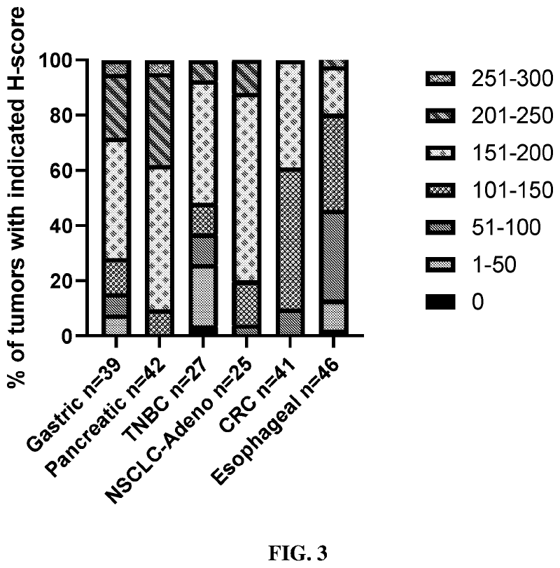

ADAM9 was highly expressed in TNBC, NSCLC, CRC, gastric, pancreatic, and esophageal cancers. A majority of the tumor samples expressed high levels of ADAM9 with 85% of gastric cancer, 100% pancreatic cancer, 63% TNBC, 96% NSCLC-adenocarcinoma, 90% CRC, 54% esophageal cancer samples having H-scores above 100 (FIG. 3). In addition, 218/220 (99%) of the cases evaluated across the indications express ADAM9 in either the membrane or cytoplasm.

a. Gastric Cancer

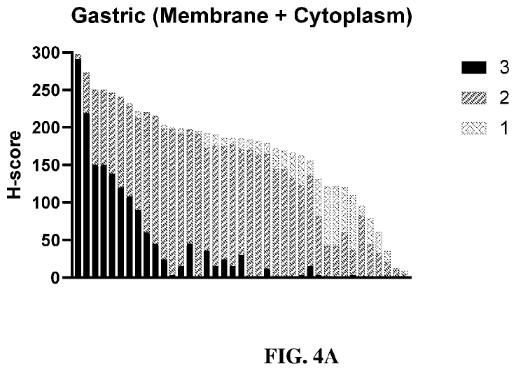

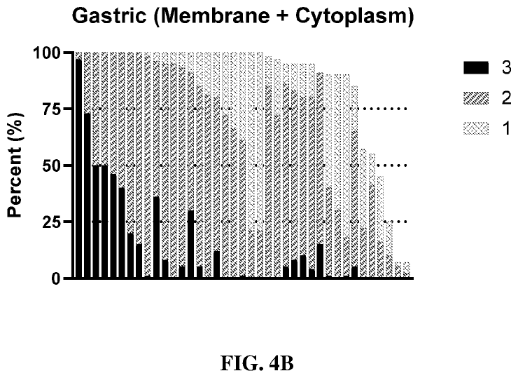

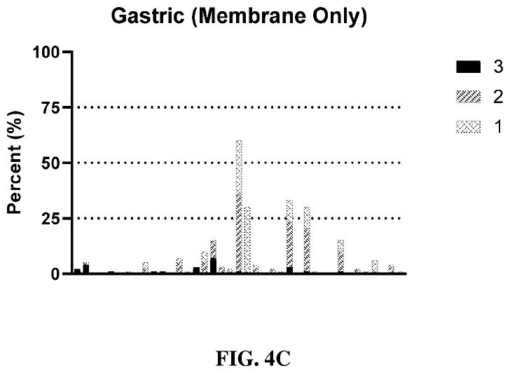

Thirty-nine whole tissue gastric cancer cases were evaluable for biomarker evaluation. In total, 39/39 cases (100%) demonstrated positive cytoplasmic or membrane staining with an average H-Score of 170 (FIGS. 4A and 4B) with 72% of samples demonstrating membrane ADAM9 staining (FIG. 4C). Positive staining is defined as any staining above background.

b. Pancreatic Cancer

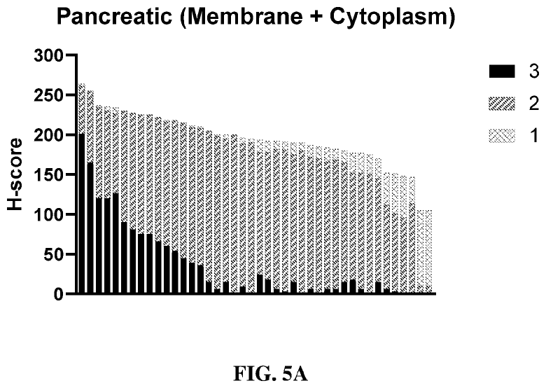

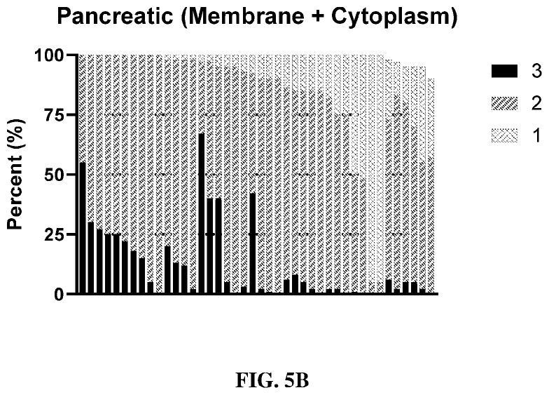

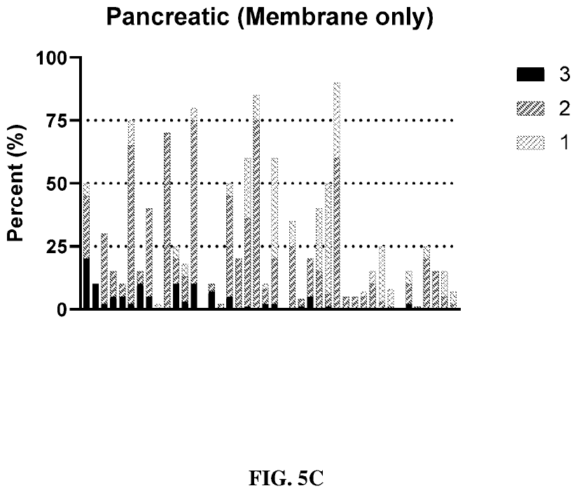

Forty-two whole tissue pancreatic cancer cases were evaluable for biomarker evaluation. In total, 42/42 (100%) demonstrated positive cytoplasmic or membrane staining, with an average H-Score of 195 (FIGS. 5A and 5B) with 93% of samples demonstrating membrane ADAM9 staining (FIG. 5C).

c. Adenocarcinoma NS CLC

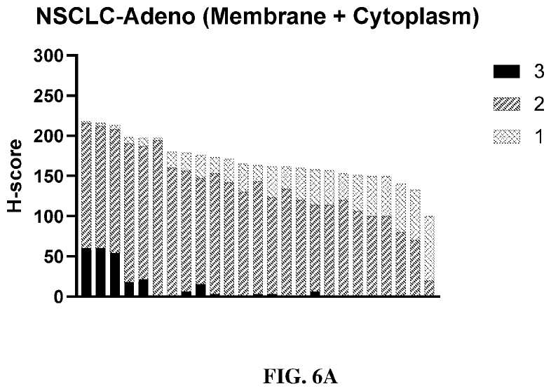

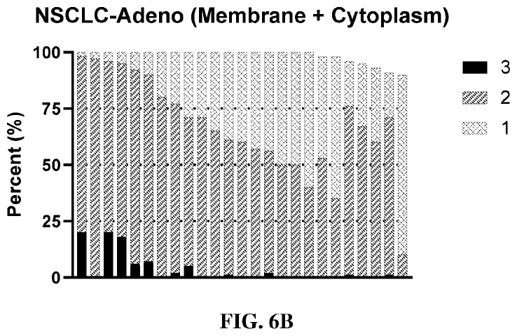

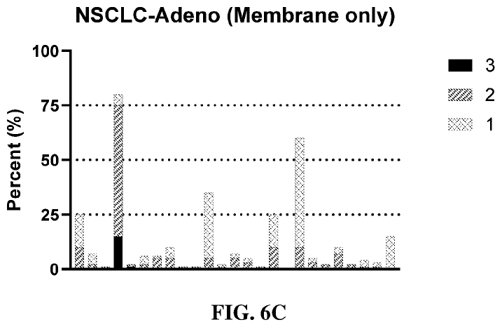

Twenty-five whole tissue NSCLC adenocarcinoma cases were evaluable for biomarker evaluation. In total, 25/25 cases (100%) demonstrated positive cytoplasm or membrane staining, with an average H-Score 169 (FIGS. 6A and 6B) with 96% of samples demonstrating membrane ADAM9 staining (FIG. 6C).

d. TNBC

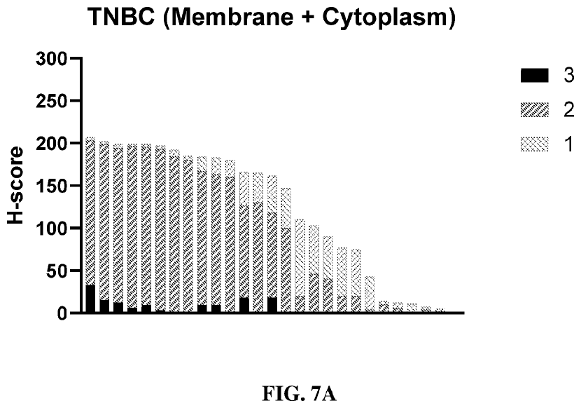

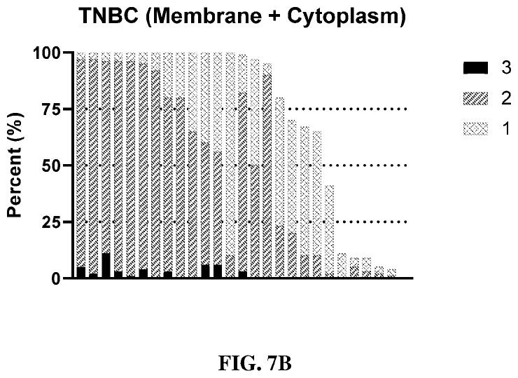

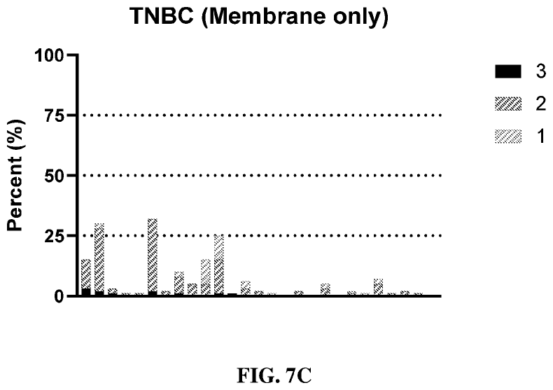

Twenty-seven whole tissue triple negative breast cancer cases were evaluable for biomarker evaluation. In total, 26/27 (96.3%) samples demonstrated positive cytoplasmic or membrane staining with an average H-Score of 123 (FIGS. 7A and 7B) with 85% of samples demonstrating membrane ADAM9 staining (FIG. 7C).

e. CRC

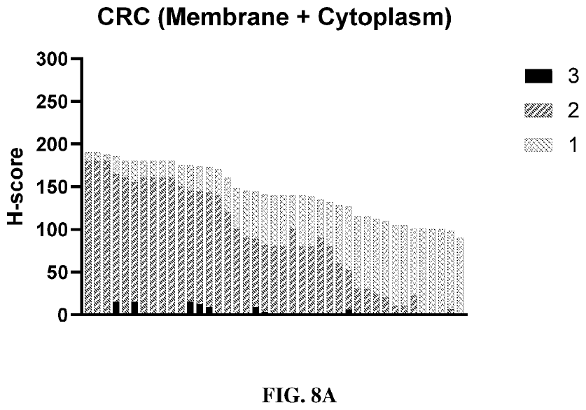

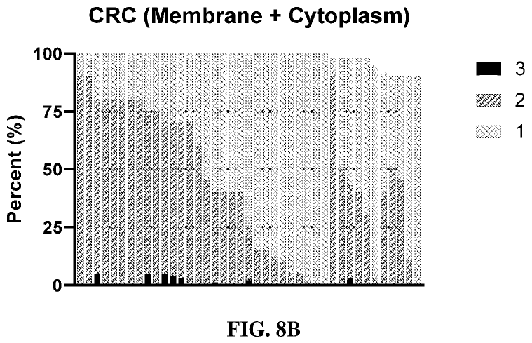

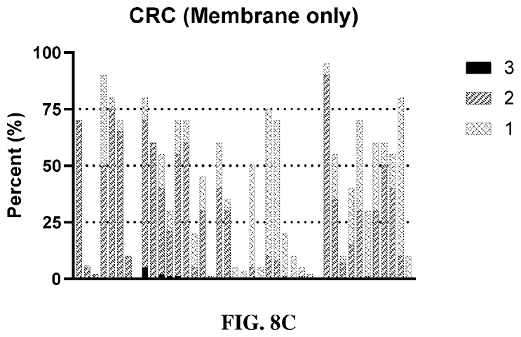

Forty-one whole tissue triple CRC cancer cases were evaluable for biomarker evaluation. In total, 41/41 (100%) samples demonstrated positive cytoplasmic or membrane staining with an average H-Score of 144 (FIGS. 8A and 8B) with 95% of samples demonstrating membrane ADAM9 staining (FIG. 8C).

f. Esophageal Cancer

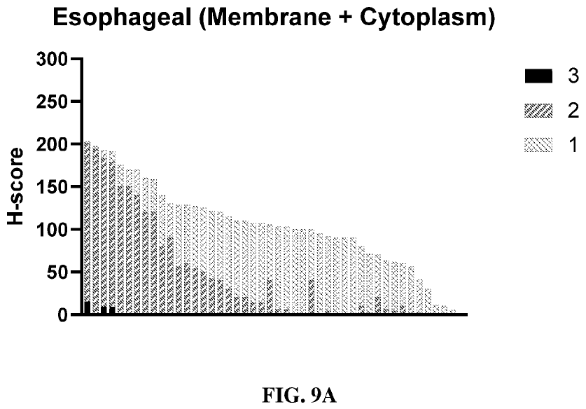

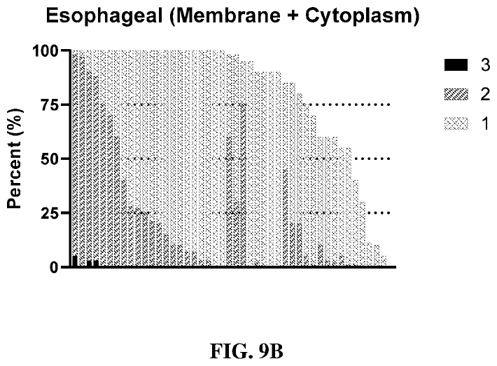

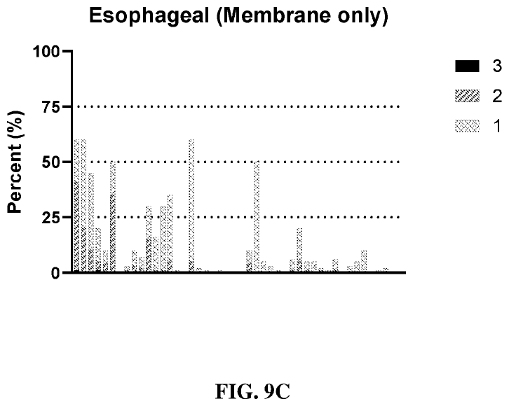

Forty-six whole tissue triple esophageal cancer cases were evaluable for biomarker evaluation. In total, 45/46 (97.8%) samples demonstrated positive cytoplasmic or membrane staining with an average H-Score of 105 (FIGS. 9A and 9B) and 76% of samples demonstrated positive ADAM9 membrane only staining (FIG. 9C).

### Example 3. ADAM9 Expression Level Assessments in a Phase I Clinical Study

A Phase 1, First-in-Human, Open-Label, Dose-Escalation Study of IMGC936 in Patients with Advanced Solid Tumors will be conducted to characterize the safety, tolerability, PK, pharmacodynamics, immunogenicity, and preliminary antitumor activity of IMGC936 by IV influsion. Participants with unresectable, relapsed or refractory, locally advanced or metastatic non-squamous NSCLC, TNBC, CRC, gastroesophageal cancer, or pancreatic cancer will be enrolled. Tumor specimens for determination of ADAM9 expression via IHC staining will be collected from all participants and will be assayed.

Analysis of ADAM9 experssion on archival and/or fresh pre-treatment harvested tumor specimens will be conducted. Participants will have an identified archival tumor specimen block (e.g., FFPE) or unstained slides from archival tumor specimen or contemporaneous tumor biopsy for the evaluation of ADAM9 expression. Participants may undergo a fresh tumor biopsy to obtain a specimen for testing if a suitable sample cannot be identified.

ADAM9 expression of participants will be analyzed to determine correlation between antitumor activity of IMGC936 and ADAM9 expression. Tumor Proportion Score (TPS) can be used to indicate positive or negative staining of ADAM9 in patient samples. TPS is calculated as percentage of viable tumor cells showing partial or complete membrane staining and cytopolasmic staining at any intensity. TPS score can also be calculated for membrane staining only, i.e., percentage of viable tumor cells showing partial or complete membrane staining only at any intensity. A reference sample from a healthy subject can be used as a baseline control or negative control. The reference sample can be from a tissue in the healthy subject that corresponds to the tissue from which the tumor specimens of the participant are collected. The reference sample can also be from a corresponding healthy tissue from the participant. In addition, or alternatively, a reference sample from a tumor tissue that expresses ADAMS can be used as a positive control.

We claim:

1. A method of treating cancer in a subject in need of the treatment, comprising administering a therapeutically effective amount of an anti-ADAM9 immunoconjugate to the subject, wherein a tumor sample from the subject exhibits an increased level of ADAM9 expression.

2. A method of increasing the efficacy of cancer treatment in a subject in need of the treatment, comprising administering a therapeutically effective amount of an anti-ADAM9 immunoconjugate to the subject, wherein a tumor sample from the subject exhibits an increased level of ADAM9 expression.

3. The method of claim 1 or 2, wherein the increased level of ADAM9 expression is determined using a detection method that distinguishes between staining intensity and/or staining uniformity in a ADAM9 expressing tumor sample as compared to staining intensity and/or staining uniformity in one or more reference samples.

4. The method of claim 3, wherein the detection method is immunohistochemistry (IHC).

5. The method of any one of claims 1-4, wherein the tumor sample is a formalin fixed paraffin embedded sample.

6. The method of any one of claims 3-5, wherein the staining intensity and/or staining uniformity is determined for ADAM9 expression in cytoplasm only, membrane only or a combination of cytoplasm and membrane (cyto-membrane) of the tumor cells.

7. The method of claim 6, wherein the staining intensity and/or staining uniformity is determined only for membrane of the tumor cells.

8. The method of any one of claims 1-7, wherein the tumor sample has a H-score between 50 and 300.

9. The method of any one of claims 1-7, wherein the tumor sample has a H-score between 100 and 300.

10. The method of any one of claims 1-7, wherein the tumor sample has a high ADAM9 expression level with a H-score between 201 and 300.

11. The method of any one of claims 1-7, wherein the tumor sample has medium ADAM9 expression level with a H-score between 101 and 200.

12. The method of any one of claims 1-7, wherein the tumor sample has a low ADAM9 expression level with a H-score between 1 and 100.

13. The method of any one of claims 1-12, wherein the tumor sample has an IHC staining intensity score of 2 or greater for ADAM9 expression level.

14. The method of any one of claims 1-12, wherein the tumor sample has an IHC staining intensity score of 3 or greater for ADAM9 expression level.

15. The method of any one of claims 1-14, wherein the tumor sample has a staining of 25% or greater PS1.

16. The method of claim 15, wherein the tumor sample has a staining of 50% or greater PS1.

17. The method of claim 15, wherein the tumor sample has a staining of 75% or greater PS1.

18. The method of any one of claims 1-14, wherein the tumor sample has a staining of 25%-49%, 50%-74% or 75%-100% PS1.

19. The method of any one of claims 1-18, the tumor sample has a staining of 25% or greater PS2.

20. The method of claim 19, wherein the tumor sample has a staining of 50% or greater PS2.

21. The method of claim 19, wherein the tumor sample has a staining of 75% or greater PS2.

22. The method of any one of claims 1-18, wherein the tumor sample has a staining of 25%-49%, 50%-74% or 75%-100% PS2.

23. The method of any one of claims 1-22, wherein the tumor sample has a staining of 25% or greater PS3.

24. The method of claim 23, wherein the tumor sample has a staining of 50% or greater PS3.

25. The method of claim 23, wherein the tumor sample has a staining of 75% or greater PS3.

26. The method of any one of claims 1-22, the tumor sample has a staining of 25%-49%, 50%-74% or 75%-100% PS3.

27. The method of any one of claims 1-26, wherein the cancer is selected from the group consisting of lung cancer, colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer.

28. The method of claim 27, wherein the cancer is non-small-cell lung cancer (NSCLC), colorectal cancer, gastric cancer, breast cancer, or pancreatic cancer.

29. The method of claim 28, wherein the cancer is adenocarcinoma NSCLC, triple negative breast cancer (TNBC), pancreatic cancer, gastric cancer or colorectal cancer.

30. The method of any one of claims 3-29, wherein the one or more reference samples comprises tissue, cells, or cell pellets.

31. The method of claim 30, wherein the one or more reference samples is a negative reference sample.

32. The method of any one of claims 1-31, wherein the anti-ADAMS immunoconjugate is represented by the following formula: 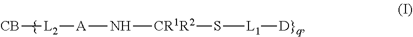
or a pharmaceutically acceptable salt thereof, wherein:
CB is an anti-ADAMS antibody or ADAMS-binding fragment thereof;
L₂ is represented by one of the following formula: 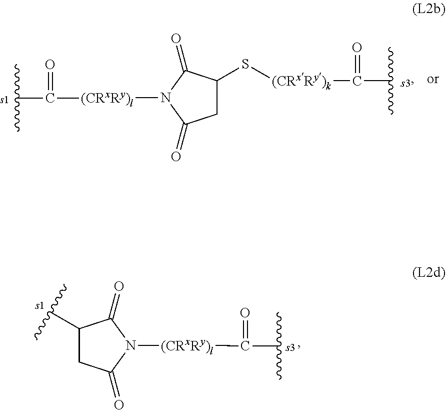
wherein:
R^{x}, R^{y}, R^{x′} and R^{y′}, for each occurrence, are independently H, —OH, halogen, —O-(C₁₋₄ alkyl), —SO₃H, —NR₄₀R₄₁R₄₂⁺, or a C₁₋₄ alkyl optionally substituted with —OH, halogen, SO₃H or NR₄₀R₄₁R₄₂⁺, wherein R₄₀, R₄₁ and R₄₂ are each independently H or a C₁₋₄ alkyl;
l and k are each independently an integer from 1 to 10;
L₁ is represented by the following formula:
—CR³R⁴—(CH₂)₁₋₈—C(═O)—
wherein R³ and R⁴ are each independently H or Me, and the —C(═O)— moiety in L₁ is connected to D;
D is represented by the following formula: 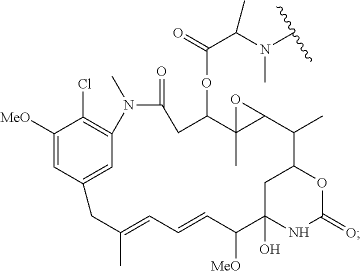 [SMILES: *N(C)C(C)C(=O)OC1CC(=O)N(C)c2cc(cc(OC)c2Cl)C/C(C)=C/C=C/C(OC)C2(O)CC(OC(=O)N2)C(C)C2OC12C]
q is an integer from 1 to 20.

33. The method of claim 32, wherein Rx, Ry, Rx′ and Ry′ are all H; and 1 and k are each independently an integer an integer from 2 to 6.

34. The method of claim 32 or 33, wherein A is a peptide containing 2 to 5 amino acid residues.

35. The method of any one of claims 32-34, wherein A is a peptide cleavable by a protease.

36. The method of any one of claims 32-34, wherein A is selected from the group consisting of Gly-Gly-Gly, Ala-Val, Val-Ala, D-Val-Ala, Val-Cit, D-Val-Cit, Val-Lys, Phe-Lys, Lys-Lys, Ala-Lys, Phe-Cit, Leu-Cit, Ile-Cit, Phe-Ala, Phe-N9-tosyl-Arg, Phe-N9-nitro-Arg, Phe-Phe-Lys, D-Phe-Phe-Lys, Gly-Phe-Lys, Leu-Ala-Leu, Ile-Ala-Leu, Val-Ala-Val, Ala-Ala-Ala, D-Ala-Ala-Ala, Ala-D-Ala-Ala, Ala-Ala-D-Ala, Ala-Leu-Ala-Leu (SEQ ID NO: 51), f3-Ala-Leu-Ala-Leu (SEQ ID NO: 52), Gly-Phe-Leu-Gly (SEQ ID NO:

53. , Val-Arg, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Gln-Val, Asn-Ala, Gln-Phe, Gln-Ala, D-Ala-Pro, and D-Ala-tBu-Gly, wherein the first amino acid in each peptide is connected to L2 group and the last amino acid in each peptide is connected to —NH—CR¹R²—S-L₁-D.

37. The method of any one of claims 32-36, wherein R 1 and R 2 are both H.

38. The method of any one of claims 32-37, wherein L₁ is —(CH₂)₄₋₆—C(50 O)—.

39. The method of any one of claims 32-38, wherein D is represented by the following formula:

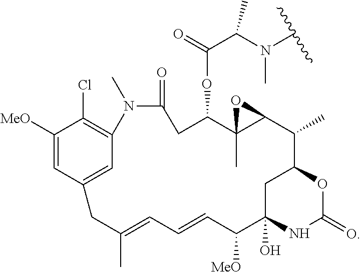 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

40. The method of any one of claims 32-39, wherein the anti-ADAMS antibody or ADAMS-binding fragment thereof is a humanized anti-ADAMS antibody or ADAM9-binding fragment thereof.

41. The method of claim 40, wherein the humanized anti-ADAMS antibody or ADAMS-binding fragment thereof comprises a CDR_{H}1 domain having the sequence of SEQ ID NO: 1, a CDR_{H}2 domain having the sequence of SEQ ID NO: 3, and a CDR_{H}3 domain having the sequence of SEQ ID NO: 14, and a CDR_{L}1 domain having the sequence of SEQ ID NO: 16, a CDR_{L}2 domain having the sequence of SEQ ID NO: 19, and a CDR_{L}3 domain having the sequence of SEQ ID NO: 20.

42. The method of claim 41, wherein the humanized anti-ADAMS antibody or ADAMS-binding fragment thereof comprises a heavy chain variable domain (VH) having the sequence of SEQ ID NO: 33 and a light chain variable domain (VL) having the sequence of SEQ ID NO: 35.

43. The method of claim 41, wherein the humanized anti-ADAMS antibody comprises a heavy chain having the sequence of SEQ ID NO: 45 and a light chain having the sequence of SEQ ID NO:50.

44. The method of claim 32, wherein the anti-ADAMS immunoconjugate is represented by the following formula: 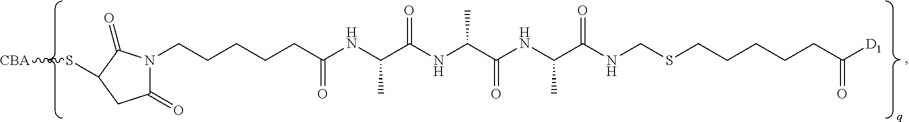
wherein:
CBA is an humanized anti-ADAMS antibody or ADAMS-binding fragment thereof comprising a CDR_{H}1 domain, a CDR_{H}2 domain, and a CDR_{H}3 domain and a CDR_{L}1 domain, a CDR_{L}2 domain, and a CDR_{L}3 domain having the sequences of SEQ ID NOs: 1, 3, and 14 and SEQ ID NOs: 16, 19, 20, respectively;
q is 1 or 2;
D₁ is represented by the following formula:

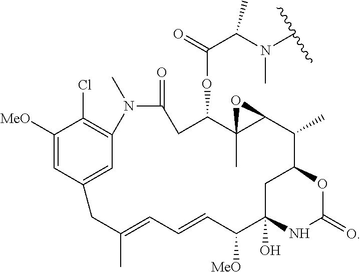 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

45. The method of claim 44, wherein the humanized anti-ADAM9 antibody or ADAM9-binding fragment thereof comprises a heavy chain variable domain (VH) and a light chain variable domain (VL) having sequences of SEQ ID NO:33 and SEQ ID NO:35, respectively.

46. The method of claim 44, wherein the humanized anti-ADAM9 antibody comprises a heavy chain and a light chain having the sequences of SEQ ID NO:45 and SEQ ID NO:50, respectively.

47. The method of claim 44, wherein the humanized anti-ADAM9 antibody comprises a heavy chain and a light chain having the sequences of SEQ ID NO:49 and SEQ ID NO:50, respectively.

48. The method of claim 44, wherein the anti-ADAM9 immunoconjugate is IMGC936.

49. A method of identifying a cancer likely to respond to an anti-ADAM9 immunoconjugate treatment, said method comprising:
(a) contacting a biological sample comprising cells from said cancer with an agent that binds ADAM9 protein of the biological sample;
(b) detecting binding of said agent that binds ADAM9 protein of said biological sample of (a);
(c) assigning a score to said binding of step (b), wherein said score is assigned based on comparison to one or more reference samples; and
(d) comparing said score in step (c) to the score of a reference tissue or cell, wherein a score for said cancer ADAM9 level that is greater than the score for a normal or low ADAM9 expressing reference sample or a score for said cancer ADAM9 level that is equal to or greater than the score for a high ADAM9 expressing reference sample identifies said cancer as likely to respond to an anti-ADAM9 immunoconjugate.

50. The method of claim 49, wherein the cancer is selected from the group consisting of lung cancer, colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer.

51. The method of claim 50, wherein the cancer is non-small-cell lung cancer (NSCLC), colorectal cancer, gastric cancer, breast cancer, or pancreatic cancer.

52. The method of claim 50, wherein the cancer is adenocarcinoma NSCLC, triple negative breast cancer (TNBC), pancreatic cancer, gastric cancer or colorectal cancer.

53. A method of identifying a tumor that is sensitive to treatment with an anti-ADAM9 immunoconjugate, said method comprising:
(a) measuring the level of ADAM9 expression in a tumor tissue sample obtained from said tumor, wherein said measuring comprises the use of a detection method that distinguishes between staining intensity or staining uniformity in a ADAM9 expressing cancer sample as compared to staining intensity or staining uniformity in one or more reference samples;
(b) determining a ADAM9 staining intensity score for said tumor tissue sample; and
(c) comparing the ADAM9 staining intensity score determined in step (b) to a relative value determined by measuring ADAM9 protein expression in at least one reference sample, wherein said at least one reference sample is a tissue, cell, or cell pellet sample which is not sensitive to treatment with anti-ADAM9 immunoconjugate, and wherein a ADAM9 staining intensity score for said sample determined in step (b) that is higher than said relative value identifies said tumor as being sensitive to the treatment.

54. The method of any one of claims 49-53, wherein said detection method is performed manually or using an automated system.

55. The method of any one of claims 49-54, wherein said detection method is IHC.

56. The method of any one of claims 49-55, wherein the sample is a formalin fixed paraffin embedded sample.

57. The method of claim 55 or 56, wherein the staining intensity and/or staining uniformity is determined for ADAM9 expression in cytoplasm only, membrane only or a combination of cytoplasm and membrane (cyto-membrane) of the tumor cells or cancer cells.

58. The method of claim 57, wherein the staining intensity and/or staining uniformity is determined only for membrane of the tumor cells or cancer cells.

59. The method of any one of claims 55-58, wherein the sample has a H-score between 50 and 300.

60. The method of any one of claims 55-58, wherein the sample has a H-score between 100 and 300.

61. The method of any one of claims 55-58, wherein the sample has a high ADAM9 expression level with a H-score between 201 and 300.

62. The method of any one of claims 55-58, wherein the sample has medium ADAM9 expression level with a H-score between 101 and 200.

63. The method of any one of claims 55-58, wherein the sample has a low ADAM9 expression level with a H-score between 1 and 100.

64. The method of any one of claims 55-63, wherein the sample has an IHC staining intensity score of 2 or greater for ADAM9 expression level.

65. The method of any one of claims 55-63, wherein the sample has an IHC staining intensity score of 3 or greater for ADAM9 expression level.

66. The method of any one of claims 55-65, wherein the tumor sample has a staining of 25% or greater PS1.

67. The method of claim 66, wherein the tumor sample has a staining of 50% or greater PS1.

68. The method of claim 66, wherein the tumor sample has a staining of 75% or greater PS1.

69. The method of any one of claims 55-65, wherein the tumor sample has a staining of 25%-49%, 50%-74% or 75%-100% PS1.

70. The method of any one of claims 55-69, wherein the tumor sample has a staining of 25% or greater PS2.

71. The method of claim 70, wherein the tumor sample has a staining of 50% or greater PS2.

72. The method of claim 70, wherein the tumor sample has a staining of 75% or greater PS2.

73. The method of claim 70, wherein the tumor sample has a staining of 25%-49%, 50%-74% or 75%-100% PS2.

74. The method of any one of claims 55-73, wherein the tumor sample has a staining of 25% or greater PS3.

75. The method of claim 74, wherein the tumor sample has a staining of 50% or greater PS3.

76. The method of claim 74, wherein the tumor sample has a staining of 75% or greater PS3.

77. The method of claim 74, wherein the tumor sample has a staining of 25%-49%, 50%-74% or 75%-100% PS3.

78. The method of any one of claims 49-77, further comprising administering a therapeutically effective amount of the anti-ADAMS immunoconjugate to a subject with the cancer or tumor.

79. The method of any one of claims 49-78, wherein the anti-ADAMS immunoconjugate is as defined in any one of claims 32-48.

80. The method of any one of claims 1-7, 27-58, 78 and 79, wherein the tumor sample has a Tumor Proportion Score (TPS) of greater or equal to 1%, greater or equal to 5%, greater or equal to 10%, greater or equal to 20%, greater or equal to 25%, greater or equal to 30%, greater or equal to 40%, greater or equal to 50%, greater or equal to 60%, greater or equal to 70%, greater or equal to 75%, greater or equal to 80%, greater or equal to 90%, or greater or equal to 95%.

81. The method of claim 80, wherein the tumor sample has a TPS of greater or equal to 25%.

82. The method of claim 80, wherein the tumor sample has a TPS of greater or equal to 50%.

83. The method of claim 80, wherein the tumor sample has a TPS of greater or equal to 75%.
